(19) United States
(12) Patent Application Publication
(10) Pub. No.: US 2017/0010272 A1
(43) Pub. Date: Jan. 12, 2017

(54) RAPID PHENOTYPE TESTS FOR ANTITUBERCULAR DRUG SENSITIVITY AND RESISTANCE

(71) Applicant: STC, UNM, Albuquerque, NM (US)

(72) Inventors: Graham Timmins, Albuquerque, NM (US); Seong Won Choi, Albuquerque, NM (US); Louis Pete A. Silks, Santa Fe, NM (US)

(21) Appl. No.: 15/119,519

(22) PCT Filed: Feb. 18, 2015
(86) PCT No.: PCT/US15/16349

§ 371 Date: Aug. 17, 2016

Related U.S. Application Data
(60) Provisional application No. 61/941,103, filed on Feb. 18, 2014.
(60) Provisional application No. 61/941,132, filed on Feb. 18, 2014.
(60) Provisional application No. 62/061,729, filed on Oct. 9, 2014.

(51) Int. Cl. G01N 33/58

(52) U.S. Cl. CPC G01N 33/58; G01N 2458/15; G01N 2333/35; G01N 2800/52

(57) ABSTRACT

In one embodiment, the invention provides methods of identifying the sensitivity and resistance to therapeutic drug regimens in a subject who suffers from, or who is suspected of suffering from, a Mycobacterium infection, the method comprising administering (1) isotopically labeled Pretomanid and/or Delaminid, or (2) isotopically labeled ethionamide and/or prothionamide, or (3) isotopically labeled pyrazinamide, or (4) isotopically-labeled isoniazid to the subject and thereafter measuring levels in a subject-derived sample of one or more isotopically-labeled markers corresponding to Mycobacterium-actiwated drug metabolites or degradation products, wherein the absence of detectable levels of Mycobacterium-activated drug metabolites or degradation products indicates either that the subject does not suffer from a Mycobacterium infection or suffers from a Mycobacterium infection which is resistant to treatment with the administered drug regimen.

### CROSS-REFERENCE TO RELATED APPLICATIONS AND GOVERNMENT SUPPORT

This application claims priority from U.S. Provisional Patent Application Ser. No. 61/941,103, entitled “Pyrazinamide Breath Test for TB”, and filed Feb. 18, 2014; U.S. Provisional Patent Application Ser. No. 61/941,132, entitled “Rapid Phenotype Test of Delaminid and PA824 Sensitivity and Resistance in Tuberculosis”, and filed Feb. 18, 2014; and U.S. Provisional Patent Application Ser. No. 62/061,729, entitled “Rapid Phenotype Test of Ethionamide and Prothionamide Resistance in Tuberculosis”, and filed Oct. 9, 2014. The complete contents of each of these provisional patent applications are hereby incorporated by reference in their entirety.

This invention was made with government support under Contract No. DE-AC52-06NA25396 awarded by the United States Department of Energy (DOE) to Los Alamos National Security, LLC, for the operation of Los Alamos National Laboratory. The government has certain rights in the invention.

### FIELD OF THE INVENTION

In one embodiment, the invention provides methods of identifying the sensitivity and resistance to therapeutic drug regimens in a subject who suffers from, or who is suspected of suffering from, a Mycobacterium infection, the method comprising administering (1) isotopically labeled Pretomanid and/or Delaminid, or (2) isotopically labeled ethionamide and/or prothionamide, or (3) isotopically labeled pyrazinamide, or (4) isotopically-labeled isoniazid to the subject and thereafter measuring levels in a subject-derived sample of one or more isotopically-labeled markers corresponding to Mycobacterium-activated drug metabolites or degradation products, wherein the absence of detectable levels of Mycobacterium-activated drug metabolites or degradation products indicates either that the subject does not suffer from a Mycobacterium infection or suffers from a Mycobacterium infection which is resistant to treatment with the administered drug regimen.

The invention therefore provides methods of rapidly diagnosing an infectious disease, monitoring therapy, and determining a bacterial phenotype in a rapid, point-of-care manner that does not require invasive sampling. In preferred embodiments, the invention provides a highly selective TB diagnostic breath test based upon a subject's Mycobacterium-activated drug metabolism.

### BACKGROUND OF THE INVENTION

Tuberculosis (TB) is an infectious disease caused by the bacillus Mycobacterium Tuberculosis (Mtb). It typically affects the lungs (pulmonary TB) but can affect other sites as well (extrapulmonary TB). The disease is spread in the air when people who are sick with pulmonary TB expel bacteria, for example by coughing. Overall, a relatively small proportion of people infected with M. tuberculosis will develop TB disease. However, the probability of developing TB is much higher among people infected with HIV. TB is also more common among men than women, and affects mainly adults in the most economically productive age groups. WHO Global Tuberculosis Report 2014.

Tuberculosis ranks as the second leading cause of death from a single infectious agent, after the human immunodeficiency virus (HIV). Around 9 million people fell ill with TB in 2013, including 1.1 million cases among people living with HIV. In 2013, 1.5 million people died from TB, including 360 000 among people who were HIV-positive. Globally, in 2013, an estimated 480,000 people developed multidrug-resistant TB (MDR-TB) and there were an estimated 210 000 deaths from MDR-TB. The number of people diagnosed with MDR-TB tripled between 2009 and 2013, and reached 136,000 worldwide. This was equivalent to 45% of the estimated MDR-TB cases among notified TB patients. Id.

Latent tuberculosis infection (LTBI) is a state of persistent immune response to stimulation by Mycobacterium tuberculosis antigens without evidence of clinically manifested active TB. A direct measurement tool for M. tuberculosis infection in humans is currently unavailable. WHO Latent Tuberculosis 2014. The following treatment regimens are currently recommended for LTBI: 6-month or 9-month isoniazid daily; 3-month rifapentine plus isoniazid weekly; 3-4 months isoniazid plus rifampicin daily; and 3-4 months rifampicin alone daily. Id.

Known vaccines against tuberculosis show very limited efficacy. The only available vaccine, Mycobacterium bovis BCG, is a highly attenuated live vaccine that exhibits limited efficacy and, due to its highly attenuated nature, proves ineffective in areas where prior environmental mycobacterial (EM) exposure has occurred. In contrast, virulent M. tuberculosis is highly infectious despite previous EM exposure. Notably, a previous M. tuberculosis infection, irrespective of treatment, does not protect against subsequent M. tuberculosis infection.

The potential of nitroimidazopyran drugs as novel drugs to treat tuberculosis (TB) was recognized in 2001¹ and led to clinical development of PA824 (Pretomanid) and OPC67683 (Delaminid).² OPC-67683 has been shown effective in otherwise drug-resistant TB³ and is in the process of receiving marketing approval, while PA824 is expected to enter approval process soon. These new drugs will be extremely important weapons in the fight against TB, especially drug-resistant TB and it is essential that their use be as judicious as possible, to prevent the rapid rise of resistance. A key tool would be a rapid assay to determine resistance or sensitivity to these drugs.

PA824 (Pretomanid) and OPC67683 (Delaminid) are prodrugs that are acted upon by a mycobacterial deazaflavin (F₄₂₀) dependent nitroreductase (Ddn) to produce nitric oxide (NO•), presumably from reduction and elimination of the nitro group, and producing des-nitro compounds (FIG. 2).^{2,4,5}

Ethionamide and prothionamide are second line TB drugs that are often used in treatment of Multi-Drug Resistant(MDR) and Extensively Drug-Resistant(XDR) TB.^{1A} They are prodrugs and are activated by the TB enzyme EthA through reactive intermediates that are not yet fully known,^{2A,3A} to form adducts of NAD that act similarly to isoniazid adducts to inhibit mycolate synthesis.^{4A} There has been significant interest in these drugs recently, as their activation and antibacterial activity can be enhanced by compounds that increase EthA expression through binding the EthA regulation protein, EthR,^{5A} and these drugs are known as boosters.^{6-8A} Most resistance to ethionamide is mediated through mutations in EthA and EthR,^{1A,9A} especially in MDR.^{10A} Interestingly, many EthA/R mutations were also associated with KatG mutation,^{9A} that could also be detected by ¹⁵N₂-INH breath test.

Several antituberculosis compounds are bioprecursor prodrugs that require activation by Mycobacterium enzymes to acquire bacterial toxicity. These include pyrazinamide (PZA), isoniazid (INH) and ethionamide (ETA) (2-ethylthioiso-nicotinamide, 2-ethylpyrimidine-4-carbothioamide). PZA is activated by the mycobacterial pyrazinamidase (PncA) to pyrazinoic acid, which lowers the pH enhancing the intracellular accumulation of the latter. Pyrazinoic acid is unable to diffuse across the mycobacterial cell wall, leading to the disruption of membrane transport and energy depletion. Because no pyrazinoic acid efflux mechanism exists, this accumulation process causes a remarkable susceptibility of M. tuberculosis to pyrazinamide. Mutations in the gene encoding pyrazinamidase/nicotinamidase (pncA) cause resistance to the antituberculous drug pyrazinamide in tubercle bacillus. Both isoniazid and the structurally analogous thioamide, ethionamide, act as inhibitors of InhA (enoyl-acyl carrier protein reductase). However, the large majority of isoniazid-resistant strains remain full susceptible to ethionamide. This is due the fact that INH and ETA are activated by different mechanisms, thus avoiding cross-resistance. Chung, et al., Prodrugs for the Treatment of Neglected Diseases, Molecules 2008, 13, 616-677 (citations omitted).

Since most resistance in mediated through altered activation, and also since booster drugs act by increasing ethionamide activation, a rapid phenotypic screen to determine ethionamide activation in the lungs of TB patients could have many uses. For example, monitoring the effect of boosters, or determining resistance rapidly.

Pyrazinamide (PZA) is one of the major drugs used to treat tuberculosis, and is a prodrug activated by tuberculosis enzymes to produce pyrazinoic acid (POA). The enzyme responsible for PZA activation is pyrazinamidase (also termed nicotinamidase) and is encoded by the pncA gene of Mycobacterium tuberculosis. See Zhang and Mitchison, “The curious characteristics of pyrazinamide: a review”, Int J Tuberc. Lung Disease 2003 7 6-21. Resistance to PZA is overwhelmingly due to mutations in the pncA gene that decrease the extent of PZA activation.

PZA resistance is increasing in the United States. See Epidemiology of Pyrazinamide-Resistant Tuberculosis in the United States, 1999-2009. Kurbatova et al. Clinical Infectious Disease, 2013 57 1081-93. Yet testing for PZA resistance is challenging. Growth-based testing for PZA resistance is difficult because the drug is only active in an acidic environment (pH ˜5.5), but this low pH itself inhibits the growth of the organism. Furthermore, even modest variations in inoculum size can alter the pH and lead to differing results, and often media containing albumin can also alter results. Zhang and Mitchison, Id. Although Next-Generation-Sequencing (NGS) can offer opportunities to identify pncA mutations that can be associated with PZA resistance (See, Next-Generation Ion Torrent Sequencing of Drug Resistance Mutations in Mycobacterium tuberculosis Strains Daum et al. Clinical Infectious Disease 2012 50 3831-87) such technology is costly, difficult to implement, and requires collection of a sample and its purification and/or culture. Furthermore, a great many mutations in pncA are found—over 300 separate mutations associated in some way with resistance are known. The correlation of genotype to phenotype (i.e. level of resistance) of these >300 mutations is very incomplete, therefore, the use of DNA sequence based resistance detection will be very difficult for PZA.

Skin tuberculin testing with purified protein derivative (PPD), is a useful first screen for potential exposure to mycobacteria but does not differentiate between prior exposure or currently active infection; chest X-rays only identify advanced lung lesions; a smear test is highly reliable but of low sensitivity since many TB patients do not present as smear positive; sputum culture of slow-growing TB bacteria is a definitive test but takes a long time and only detects active disease.

The need therefore exists for methods of rapidly diagnosing an infectious disease, monitoring therapy, and determining a bacterial phenotype in a rapid, point-of-care manner that does not require invasive sampling. This need is particularly acute, given the resistance threat posed by initially ineffective and incorrect TB diagnoses and treatments.

### SUMMARY OF THE INVENTION

We have discovered methods of rapidly diagnosing an infectious disease, monitoring therapy, and determining a bacterial phenotype in a rapid, point-of-care manner that does not require invasive sampling. Our invention provides a highly selective a Mycobacterium infection diagnostic breath test based upon a subject's Mycobacterium-activated drug metabolism. Using our invention, active diagnoses can be made rapidly at a point of care, so that treatment can begin immediately, thereby preventing further disease progression with known diagnostic modalities that require long waiting periods between sampling and diagnosis.

We have discovered that stable isotope derivatives of PA824 (Pretomanid), OPC67683 (Delaminid), ethionamide, prothionamide, pyrazinamide (PZA) and isoniazid can be used diagnostically to confirm the presence of a Mycobacterium (e.g. M. tuberculosis) infection and to ascertain the resistance and/or sensitivity of such an infection to treatment by one or more of the aforementioned drugs.

For example, we have determined that in a M. tuberculosis-infected subject, ¹⁵N-nitro-Pretomanid and ¹⁵N-nitro-Delaminid generate in vivo the M. tuberculosis metabolic/cleavage products ¹⁵NO• or ¹⁵NO degradation products (e.g. ¹⁵N-nitrate or nitrite (¹⁵NO₃⁻ or ¹⁵NO₂⁻)). We have also determined that in a M. tuberculosis-infected subject, ¹⁵N, ³³S, ³⁴S or ³⁶S-labeled ethionamide and prothionamide generate in vivo the M. tuberculosis metabolic/cleavage products ¹⁵NH₃ (where ¹⁵N-labeled drug was administered) and sulfur oxides of the formula SOₓ, where x is an integer from 2 to 4 (where ³³S, ³⁴S or ³⁶S-labeled drug was administered). Further, we have found that ¹⁵N-isotopically labeled pyrazinamide yields in vivo the M. tuberculosis metabolic/cleavage products ¹⁵NH₃ and ¹⁵N-urea. In the case of ¹³C or ¹⁷O/¹⁸O-labeled pyrazinamide, we have found that the M. tuberculosis metabolic/cleavage products are ¹³C-pyrazinoic acid or ¹⁷O/¹⁸O-pyrazinoic acid. Finally, we determined that ¹⁵N-isoniazid yields in vivo the M. tuberculosis metabolic/cleavage products ¹⁵NO and/or ¹⁵N₂. It is noted here that ¹⁵NO is not produced from pyrazinamide, ¹⁵NH₃ is not produced from PA824 or delaminid and there should be no interconversion between ¹⁵NO and ¹⁵NH₃ or vice versa.

We applied these discoveries in our invention of novel methods of diagnosing a Mycobacterium infection (e.g. a Mycobacterium tuberculosis (M. tuberculosis) infection) and its drug responsiveness and sensitivity using a simple diagnostic test. Diagnoses can be done using a breath test and are therefore rapid, accurate and offer a number of ancillary advantages described hereinafter.

In one embodiment, the invention provides methods of identifying the sensitivity and resistance to therapeutic drug regimens in a subject who suffers from, or who is suspected of suffering from, a Mycobacterium infection, the method comprising:

(a) administering ¹⁵N-nitro-PA824 (¹⁵N-nitro-Pretomanid) and/or ¹⁵N-nitro-OPC67683 (¹⁵N-nitro-Delaminid) to the subject and thereafter measuring levels in a subject-derived sample (e.g. a breath, blood, saliva, urine and/or sputum sample) of one or more markers selected from the group consisting of ¹⁵NO or ¹⁵NO degradation products (e.g. ¹⁵N-nitrate or nitrite (¹⁵NO₃⁻ or ¹⁵NO₂⁻)), wherein detectable levels in the sample of ¹⁵NO• or ¹⁵NO degradation products indicate that the subject suffers a Mycobacterium infection which is susceptible to treatment with PA824 (Pretomanid) and/or OPC67683 (Delaminid), and wherein the absence of detectable levels of ¹⁵NO• or ¹⁵NO degradation products indicates either that the subject does not suffer from a Mycobacterium infection or suffers from a Mycobacterium infection which is resistant to treatment with PA824 (Pretomanid) and/or OPC67683 (Delaminid); and/or
(b) administering (1) ¹⁵N-ethionamide, or a sulfur isotope-labeled ethionamide selected from the group consisting of ³³S-ethionamide, ³⁴S-ethionamide or ³⁶S-ethionamide and/or (2) ¹⁵N-prothionamide or a sulfur isotope-labeled prothionamide selected from the group consisting of ³³S-prothionamide, ³⁴S-prothionamide or ³⁶S-prothionamide to the subject and thereafter measuring levels in a subject-derived sample (e.g. a breath, blood, saliva, urine and/or sputum sample) of (i)¹⁵NH₃ if ¹⁵N-ethionamide or ¹⁵N-prothionamide was administered to the subject, and/or (ii) sulfur oxides of the formula SOₓ, where x is an integer from 2 to 4, if a sulfur isotope-labeled ethionamide or sulfur isotope-labeled prothionamide was administered to the subject, wherein detectable levels in the sample of ¹⁵NH₃ or sulfur oxides indicate that the subject suffers a Mycobacterium infection which is susceptible to treatment with ethionamide and/or prothionamide, and wherein the absence of detectable levels of ¹⁵NH₃ or sulfur oxides indicate either that the subject does not suffer from a Mycobacterium infection or suffers from a Mycobacterium infection which is resistant to treatment with ethionamide and/or prothionamide; and/or
(c) administering ¹⁵N-pyrazinamide (PZA) and/or ¹³C-pyrazinamide and/or an oxygen isotope-labeled pyrazinamide selected from the group consisting of ¹⁷O-pyrazinamide and ¹⁸O-pyrazinamide to the subject and thereafter measuring levels in a subject-derived sample (e.g. a breath, blood, saliva, urine and/or sputum sample) of ¹⁵NH₃ and/or ¹⁵N-urea if ¹⁵N-pyrazinamide (PZA) was administered to the subject, and/or ¹³C-pyrazinoic acid if ¹³C-pyrazinamide was administered to the subject, and/or ¹⁷O-pyrazinoic acid or ¹⁸O-pyrazinoic acid if an oxygen isotope-labeled pyrazinamide was administered to the subject, wherein detectable levels in the sample of ¹⁵NH₃, ¹³C-pyrazinoic acid, and/or ¹⁷O-pyrazinoic acid or ¹⁸O-pyrazinoic acid indicate that the subject suffers from a Mycobacterium infection which is susceptible to treatment with pyrazinamide, and wherein the absence of detectable levels of ¹⁵NH₃, ¹³C-pyrazinoic acid, and/or ¹⁷O-pyrazinoic acid or ¹⁸O-pyrazinoic acid indicate either that the subject does not suffer from a Mycobacterium infection or suffers from a Mycobacterium infection which is resistant to treatment with pyrazinamide and/or
(d) administering ¹⁵N-isoniazid to the subject and thereafter measuring levels in a subject-derived sample of ¹⁵NO and/or ¹⁵N₂, wherein detectable levels in the sample of ¹⁵NO and/or ¹⁵N₂ indicate that the subject suffers from a Mycobacterium infection which is susceptible to treatment with isoniazid, and wherein the absence of detectable levels of ¹⁵NO and/or ¹⁵N₂ indicate either that the subject does not suffer from a Mycobacterium infection or suffers from a Mycobacterium infection which is resistant to treatment with isoniazid.

In a preferred embodiment, the invention provides a method for diagnosing a M. tuberculosis infection in the lungs (or other tissues) in accordance with the methods described above. Diagnostic methods of the invention may optionally include the step(s) of analyzing an isotopic cleavage product or metabolite plasma concentration-time curve (e.g. determining the area under the curve (AUC)) to determine the extent of resistance.

Preferably, isotopic cleavage products or metabolites are detected in the exhaled breath of a subject, and breath samples are taken repeatedly at regular intervals (e.g. ranging from several breaths in a short period of time to a number of breaths analyzed over the course of several minutes or more). The predetermined measurement period may be determined by analyzing the drug metabolism of M. tuberculosis-control subjects (e.g. AUC analyses of isotopically labeled cleavage products and/or metabolites found in the exhaled breath of the control subjects). Analysis of a subject's exhalations enable an assessment of the presence and potential drug resistance of a M. tuberculosis infection. In certain embodiments, the step of analyzing the exhaled breath of the subject is repeated substantially until a particular accuracy for analyzing the data is reached.

Variation of concentration ratios of isotopically labeled element: non-isotopically labeled element concentration ratios (y-axis) over time (x-axis) can also be plotted and analyzed by techniques which are well-known to those of ordinary skill in the art (e.g. calculation plasma profile area under the curve (AUC)). In certain embodiments, the breath of the subject is analyzed by an infrared laser spectrometer or a mass spectrometer to determine concentrations of isotopically labeled elements and non-isotopically labeled elements.

In another preferred embodiment, isotopically labeled Pretomanid and/or Delaminid, or isotopically labeled ethionamide and/or prothionamide, or isotopically labeled pyrazinamide, or isotopically labeled isoniazid (optionally in combination with a booster drug) is/are administered by inhalation (e.g. using a nebulizer, dry powder inhaler, or instillation), and the levels of isotopically labeled cleavage products/metabolites are measured in the subject's breath both before administration of labeled drug and at time points thereafter. Measuring increases in cleavage products/metabolites shortly after administration (e.g., about 1-5 minutes, about 5-10 minutes, about 15-20 minutes, about 25-30 minutes, about 35-40 minutes, about 45-50 minutes, about 55-60 minutes or at regular intervals over the course of about 1, 2, 3, 4 or 5 hours) better predicts drug activation by lung Mycobacteria and provides a more accurate indicator of drug sensitivity than comparable measurements taken at a relatively extended time period after drug administration. This is because over increasing lengths of time, generation of products/metabolites such as ¹⁵NH₃ may be attributable either to Mycobacteria or other non-infection metabolic pathways (e.g. hepatic microsomal deamidase metabolism of pyrazinamide generates peak metabolite levels around six hours after hours after oral dosage pyrazinamide).

In one embodiment, ¹⁵N-amide labeled PZA is administered by inhalation (e.g. using a nebulizer, dry powder inhaler, or instillation), and the levels of ¹⁵NH₃ are measured in breath both before labeled PZA administration, and at time points thereafter. Increases in ¹⁵NH₃ shortly after administration indicate PZA activation by lung Mycobacteria, and are predictive of PZA sensitivity. See FIG. 1B (amide-15N-pyrazinamide (¹⁵N-PZA) and its conversion to ¹⁵NH₃ by M. tuberculosis).

In another embodiment, levels of ¹⁵NO• or its nitrate and/or nitrite metabolites in are measured in the breath, blood, saliva, urine of a patient who has been administered ¹⁵N-labeled PA824 and/or OPC67683 and who suffers from, or who is suspected of suffering from, a Mycobacterium infection. Evidence of ¹⁵NO in the breath of the patient is evidence that are he or she suffers from a Mycobacterium infection which is responsive to treatment with PA824/OPC67683, and the absence of labeled ¹⁵NO• in the breath of the patient is evidence that a Mycobacterium infection is either absent or resistant to PA824/OPC67683.

Our invention also enables the use of BCG vaccination without compromising the sensitivity or reliability of the tuberculosis test. By avoiding reliance on a PPD skin test, it enables BCG vaccination without compromising a sensitive, specific, and reliable detection of TB infection. Further, our novel diagnostic methods are one-step, readily administered tests, unlike the skin tuberculin test which requires expert administration and post-diagnostic follow-up. For example, administration of a tracer tablet and collecting breath or urine afterwards requires a low level of skill and can be administered by patients or paramedics, and test samples may be forwarded readily to remote analysis sites. In another example, foreign-deployed military personnel who test positive in a non-specific skin tuberculin test post-deployment are often administered a 6 to 12 month course of isoniazid. If the patient is not actually infected with TB, such prolonged, unnecessay use of isoniazid can prove hepatotoxic. Our methods are specific and minimize such unnecessary chemoprophylaxis and associated side effects. Also, our methods can track levels of KatG mediated INH-derived volatiles. INH resistance is typically associated with mutations affecting mycobacterial peroxidase KatG. Thus, upon resistance, the amount of KatG mediated INH-derived volatiles will change, and so development of INH resistance can also be monitored with this technique.

The invention also provides compositions selected from the group consisting of:

(a) ¹⁵N-nitro-PA824 (¹⁵N-nitro-Pretomanid) and ¹⁵N-nitro-OPC67683 (¹⁵N-nitro-Delaminid);
(b) ¹⁵N-ethionamide; ³³S-ethionamide, ³⁴S-ethionamide and ³⁶S-ethionamide;
(c) ¹⁵N-prothionamide, ³³S-prothionamide, ³⁴S-prothionamide and ³⁶S-prothionamide;
(d) ¹⁵N-pyrazinamide, ¹³C-pyrazinamide, ¹⁷O-pyrazinamide and ¹⁸O-pyrazinamide; and

### (e) ¹⁵N-isoniazid.

The novel compositions ¹⁵N-nitro-PA824 (¹⁵N-nitro-Pretomanid) and/or ¹⁵N-nitro-OPC67683 (¹⁵N-nitro-Delaminid) and related syntheses are described in FIG. 3. Certain embodiments of these syntheses are variations of techniques described in Marsini, et al., The Journal of Organic Chemistry 75, 7479-7482, doi:10.1021/jo1015807 (2010). For example, ¹⁵N-dinitroimidazole or ¹⁵N-chloro-nitro-imidazole are prepared and used to make ¹⁵N-nitro-PA824 (¹⁵N-nitro-Pretomanid) and/or ¹⁵N-nitro-OPC67683 (¹⁵N-nitro-Delaminid).

The novel compositions ¹⁵N-pyrazinamide and/or ¹³C-pyrazinamide and related syntheses are described in FIG. 1B. ¹⁵N-pyrazinamide can be made by heating pyrazine 2, 3-dicarboxylic acid with ¹⁵N₂-urea and collecting the resulting ¹⁵N-pyrazinamide by sublimation.

Kits of the invention can comprise isotopically labeled Pretomanid and/or Delaminid, or isotopically labeled ethionamide and/or prothionamide, or isotopically labeled pyrazinamide, or isotopically labeled isoniazid in oral or pulmonary dosage form, a collection bag or vial to collect exhaled breaths from a subject, and an optional instruction manual.

Diagnostic methods of the invention are practical, sensitive and specific; their results are not influenced by stress, exercise, hormone imbalances, or medications and they are non-invasive method. Our diagnostic methods are simple to perform and can be readily used in physicians' offices or medical laboratories. They employ stable isotopes (e.g. ¹⁵N) which occur naturally and are ubiquitous. Use of stable isotopes in our diagnostic methods facilitates their use in children and women of child-bearing age.

These and other aspects are described further in the Detailed Description of the Invention.

### BRIEF DESCRIPTION OF THE FIGURES

FIG. 1 shows the chemical structures of Delamanid and PA824.

FIG. 2 shows the activation of PA824 and OPC67683 to NO• by Ddn.

FIG. 3 shows the chemical structures of ¹⁵N-nitro-OPC67683 and ¹⁵N-nitro-PA824 FIG. 4 shows the Ddn mediated production of ¹⁵NO from ¹⁵N-nitro-OPC67683 and ¹⁵N-nitro-PA824.

FIGS. 5A and 5B show the chemical synthesis of ¹⁵N-nitro-PA824. In FIG. 5A, ¹⁵N-nitro-PA824 is synthesized from 2-¹⁴N,4-¹⁵N-dinitroimidazole glycidyl t-butyl dimethyl silane. In FIG. 5B, ¹⁵N-nitro-PA824 is synthesized from 2-Chloro-4-¹⁵N-nitroimidazole (chloronitroimidazole) and (R)-3-Chloro-2-(4-(trifluoromethoxy)benzyloxy)propyl 4-Methoxybenzoate.

FIG. 6 shows the chemical synthesis of ¹⁵N-nitro-delamanid starting from chloronitroimidazole.

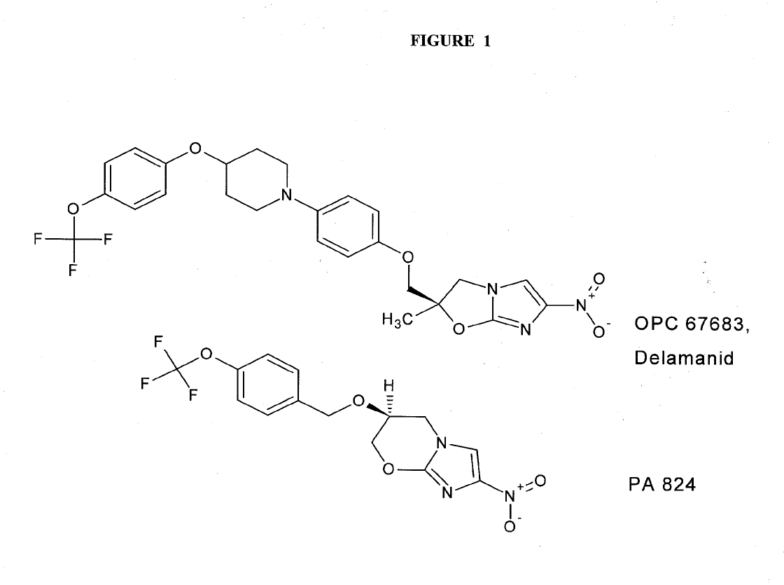

FIG. 1A shows sulfur and/or nitrogen isotope (thioamide) labeled ethionamide and prothionamide and its conversion to isotopically labeled ammonia (preferably, ¹⁵NH₃) and/or sulfur (³³S, ³⁴S or ³⁶S) chemical species by M. tuberculosis.

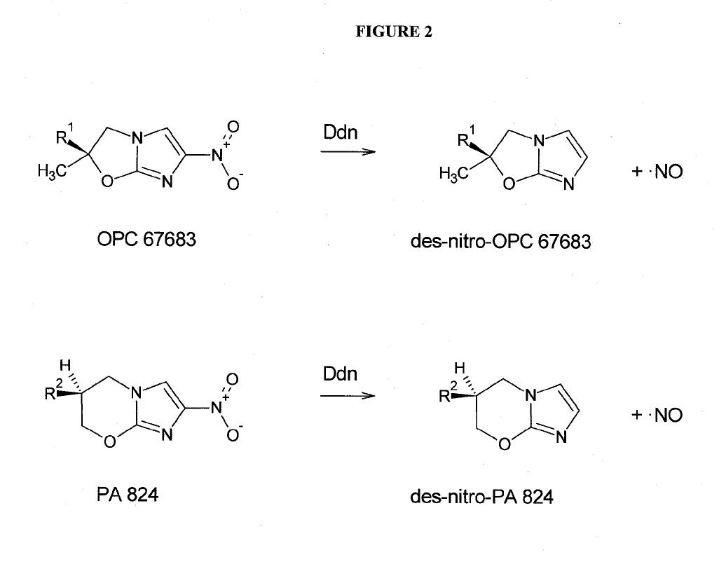

FIG. 2A shows the chemical synthesis of ethionamide and prothionamide from an appropriately substituted ethylisonicotinate analog with labeled nitrogen or sulfur as previously described by Liberman, U.S. Pat. No. 2,901,488, which is incorporated by reference in its entirety herein.

FIG. 1B shows amide-¹⁵N-pyrazinamide (¹⁵N-PZA) and its conversion to ¹⁵NH₃ by M. tuberculosis.

### DETAILED DESCRIPTION OF INVENTION

The following terms are used to describe the present invention. In the event that a term is not specifically defined herein, that term is accorded its commonly understood meaning within the context of its use by those of ordinary skill in the art. It is understood that the definitions of the terms which are used to describe the present invention are interpreted in a manner consistent with the present invention and within the context of a particular term's use in describing the present invention.

As used herein and in the appended claims, the singular forms “a,” “an,” and “the” include plural reference unless the context clearly dictates otherwise. Thus, for example, a reference to “an isotopically labeled element” includes a plurality (for example, two or more elements) of such elements, and so forth. Under no circumstances is the patent be interpreted to be limited to the specific examples or embodiments or methods specifically disclosed herein.

The term “patient” or “subject” is used to describe an individual subject or patient, a mammal, generally a human, who has been exposed to tuberculosis, is suspected of having been exposed to tuberculosis or is to be diagnosed for possible exposure to tuberculosis using one or more methods according to the present invention.

The term “effective” refers to an amount of a substrate (e.g. isotopically labeled Pretomanid and/or Delaminid, or isotopically labeled ethionamide and/or prothionamide, or isotopically labeled pyrazinamide or isotopically labeled isoniazid) which is sufficient to produce a detectable level of a cleavage product or products, without an untoward level of adverse side effects, such as toxicity, irritation, allergy or hypersensitivity responses. The level of any such side effects should be commensurate with acceptable risk/benefit ratios. In the present invention, the term effective is used to describe an amount of a substrate or other substance which is used to effect an intended result within the context of its use.

As applied to ethionamide and/or prothionamide, the term “booster drug”, “ethionamide/prothionamide booster” or “EthA inducer” is used to describe a compound which enhances the activity of ethionamide/prothionamide by binding the transcriptional repressor of EthA, known as EthR, inhibiting repression and consequently, boosting levels of the enzyme EthA. Examples of booster drugs for use in the present invention include, for example:

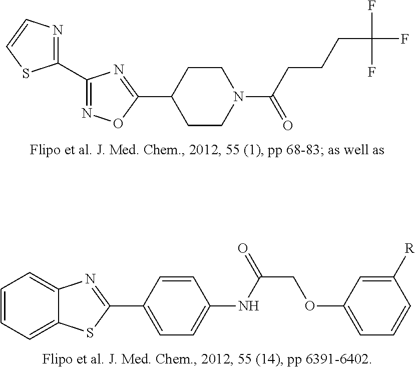

The term “co-administration” is used to describe the administration of two active compounds. Although the term co-administration preferably includes the administration of two active compounds to the patient at the same time, it is not necessary that the compounds actually be administered at the exact same time, only that amounts of compound will be administered to a patient or subject such that effective concentrations are found in the blood, serum or plasma, or in the pulmonary tissue at the same time.

Isotopically labeled Pretomanid and/or Delaminid, or isotopically labeled ethionamide and/or prothionamide, or isotopically labeled pyrazinamide, or isotopically labeled isoniazid, after exposure to a Mycobacterium (e.g. sensitive M. tuberculosis), generate isotopically-labeled cleavage products or metabolites indicative of the infection's responsiveness to treatment with the aforementioned drug regimens. Absence of cleavage products indicates that the M. tuberculosis infection is resistant to treatment with Pretomanid and/or Delaminid, or ethionamide and/or prothionamide, or pyrazinamide, or isoniazid. For example, presence or absence of isotopically labeled ammonia, urea and/or pyrazinoic acid is indicative of a M. tuberculosis infection sensitivity or resistance to treatment with pyrazinamide.

“Mycobacterium infections” are infections caused by intracellular microorganisms of the genus Mycobacterium, including diseases caused by the species M. tuberculosis, M. africanum, M. bovis, M. bovis BCG, M. canetti, M. microti, M. caprae, M. pinnipedii, M. avium, and M. leprae. “Mycobacterium infections” include infections caused by members of the Mycobacterium tuberculosis complex, the Mycobacterium avium complex, the Mycobacterium gordonae clade, the Mycobacterium kansasii clade, the Mycobacterium nonchromogenicum/terrae clade, the Mycolactone-producing mycobacteria, the Mycobacterium simiae clade, the Mycobacterium chelonae clade, the Mycobacterium fortuitum clade, the Mycobacterium parafortuitum clade and the Mycobacterium vaccae clade.

“Mycobacterium infections” include infections associated with nontuberculous mycobacteria (NTM), which are classified based on their growth rates. Rapidly growing NTM are categorized into pigmented and nonpigmented species. Mycobacterium fortuitum complex is nonpigmented and includes the M. fortuitum group and the Mycobacterium chelonae/abscessus group. The pigmented species are rarely associated in clinical disease and include Mycobacterium phlei, Mycobacterium aurum, Mycobacterium flavescens, Mycobacterium vaccae, Mycobacterium neoaurum, and Mycobacterium thermoresistible. Mycobacterium smegmatis may be either pigmented or nonpigmented.

“Mycobacterium infections” also include atypical mycobacterial infections. Mycobacterium avium complex (MAC) and Mycobacterium scrofulaceum are associated with lymphadenitis in immunocompetent children. MAC has also been associated with the pulmonary infection and bronchiectasis in elderly women without a preexisting lung disease. Pulmonary MAC infection in this population is believed to be due to voluntary cough suppression that results in stagnation of secretions, which is suitable for growth of the organisms. Mycobacterium ulcerans, the agent of a chronic ulcerative skin infection called Buruli ulcer, is widespread in Ghana, Cote d'Ivoire, Senegal, Uganda, and most central African countries. Medscape, Atypical Mycobacterial Infection.

The term “Tuberculosis” or “TB” is used to describe the infection caused by the infective agent “Mycobacterium tuberculosis” or “M. tuberculosis”, a tubercle bacillus bacteria. Tuberculosis is a potentially fatal contagious disease that can affect almost any part of the body but is most frequently an infection of the lungs. It is caused by a bacterial microorganism, the tubercle bacillus or Mycobacterium tuberculosis.

Tuberculosis is primarily an infection of the lungs, but any organ system is susceptible, so its manifestations may be varied. Effective therapy and methods of control and prevention of tuberculosis have been developed, but the disease remains a major cause of mortality and morbidity throughout the world. The treatment of tuberculosis has been complicated by the emergence of drug-resistant organisms, including multiple-drug-resistant tuberculosis, especially in those with HIV infection. M. tuberculosis is transmitted by airborne droplet nuclei produced when an individual with active disease coughs, speaks, or sneezes. When inhaled, the droplet nuclei reach the alveoli of the lung. In susceptible individuals the organisms may then multiply and spread through lymphatics to the lymph nodes, and through the bloodstream to other sites such as the lung apices, bone marrow, kidneys, and meninges.

The development of acquired immunity in 2 to 10 weeks results in a halt to bacterial multiplication. Lesions heal and the individual remains asymptomatic. Such an individual is said to have a tuberculosis infection without disease, and will show a positive tuberculin test. The risk of developing active disease with clinical symptoms and positive cultures for the tubercle bacillus diminishes with time and may never occur, but is a lifelong risk. Approximately 5% of individuals with a tuberculosis infection progress to active disease. Progression occurs mainly in the first 2 years after infection; household contacts and the newly infected are thus at risk.

Many of the symptoms of tuberculosis, whether pulmonary disease or extrapulmonary disease, are nonspecific. Fatigue or tiredness, weight loss, fever, and loss of appetite may be present for months. A fever of unknown origin may be the sole indication of tuberculosis, or an individual may have an acute influenza-like illness. Erythema nodosum, a skin lesion, is occasionally associated with the disease.

The lung is the most common location for a focus of infection to flare into active disease with the acceleration of the growth of organisms. Infections in the lung are the primary focus of the present invention. There may be complaints of cough, which can produce sputum containing mucus, pus- and, rarely, blood. Listening to the lungs may disclose rales or crackles and signs of pleural effusion (the escape of fluid into the lungs) or consolidation if present. In many, especially those with small infiltration, the physical examination of the chest reveals no abnormalities.

Miliary tuberculosis is a variant that results from the blood-borne dissemination of a great number of organisms resulting in the simultaneous seeding of many organ systems. The meninges, liver, bone marrow, spleen, and genitourinary system are usually involved. The term miliary refers to the lung lesions being the size of millet seeds (about 0.08 in. or 2 mm). These lung lesions are present bilaterally. Symptoms are variable.

Extrapulmonary tuberculosis is much less common than pulmonary disease. However, in individuals with AIDS, extrapulmonary tuberculosis predominates, particularly with lymph node involvement, with some pulmonary impact. For example, fluid in the lungs and lung lesions are other common manifestations of tuberculosis in AIDS. The lung is the portal of entry, and an extrapulmonary focus, seeded at the time of infection, breaks down with disease occurring.

Development of renal tuberculosis can result in symptoms of burning on urination, and blood and white cells in the urine; or the individual may be asymptomatic. The symptoms of tuberculous meningitis are nonspecific, with acute or chronic fever, headache, irritability, and malaise.

A tuberculosis pleural effusion can occur without obvious lung involvement. Fever and chest pain upon breathing are common symptoms. Bone and joint involvement results in pain and fever at the joint site. The most common complaint is a chronic arthritis usually localized to one joint. Osteomyelitis is also usually present. Pericardial inflammation with fluid accumulation or constriction of the heart chambers secondary to pericardial scarring are two other forms of extrapulmonary disease.

At present, the principal methods of diagnosis for pulmonary tuberculosis are the tuberculin skin test (an intracutaneous injection of purified protein derivative tuberculin is performed, and the injection site examined for reactivity), sputum smear and culture, and the chest x-ray. Culture and biopsy are important in making the diagnosis in extrapulmonary disease.

A combination of two or more drugs is often used in the initial therapy of tuberculosis disease. Drug combinations are used to lessen the chance of drug-resistant organisms surviving. The preferred treatment regimen for both pulmonary and extrapulmonary tuberculosis is a 6-month regimen of the antibiotics isoniazid, rifampin, and pyrazinamide given for 2 months, followed by isoniazid and rifampin for 4 months. Because of the problem of drug-resistant cases, ethambutol can be included in the initial regimen until the results of drug susceptibility studies are known. Once treatment is started, improvement occurs in almost all individuals. Any treatment failure or individual relapse is usually due to drug-resistant organisms.

The invention provides diagnostic pharmaceutical compositions comprising isotopically labeled Pretomanid and/or Delaminid, or isotopically labeled ethionamide and/or prothionamide, or isotopically labeled pyrazinamide, or isotopically labeled isoniazid. When a pharmaceutical composition of the invention is used in powder form, the powder mean particle diameter is preferably within the range of about 0.1 to 20 μm, and a range of about 1 to 5 μm is particularly preferred. Preferably, particles having a size of about 25 μm or more account for not more than about 5% of the particles, and preferably, 1% or less to maximize isotopically labeled drug delivery into the lungs of the subject.

The diagnostic pharmaceutical composition in the form of a powder of the invention can be produced by drying-micronization, spray drying and other standard pharmaceutical methodologies that are well-known to those of ordinary skill in the art.

By way of example, using drying-pulverization techniques, a pharmaceutical composition in the form of a powder can be prepared by drying an aqueous solution (or aqueous dispersion) containing isotopically labeled Pretomanid and/or Delaminid, or isotopically labeled ethionamide and/or prothionamide, or isotopically labeled pyrazinamide, or isotopically labeled isoniazid, along with excipients which provide for immediate release in pulmonary tissue. Dried product is then microparticulated. More specifically, after dissolving (or dispersing) a pharmaceutically acceptable carrier, additive or excipient in an aqueous medium, diagnostically (therapeutically) effective amounts of isotopically labeled Pretomanid and/or Delaminid, or isotopically labeled ethionamide and/or prothionamide, or isotopically labeled pyrazinamide, or isotopically labeled isoniazid are added and dissolved (or dispersed) by stirring (e.g. using a homogenizer) to yield an aqueous solution (or aqueous dispersion). The aqueous medium may be water or a mixture of water and a lower alcohol (e.g. methanol, ethanol, 1-propanol, 2-propanol and similar water-miscible alcohols; ethanol is particularly preferred). The resultant aqueous solution (or aqueous dispersion) is dried (e.g. by blower, lyophilization, etc.), and the resulting product is pulverized or microparticulated into fine particles (e.g. using jet mills or ball mills) to yield a powder having a mean particle diameter as described above. If necessary, additives may be added as needed in any of the above steps.

According to the spray-drying method, the pharmaceutical composition in the form of a powder of the invention can be prepared, for example, by spray-drying an aqueous solution (or aqueous dispersion) containing isotopically labeled Pretomanid and/or Delaminid, or isotopically labeled ethionamide and/or prothionamide, or isotopically labeled pyrazinamide, or isotopically labeled isoniazid and excipients, addtives or carriers for microparticulation. The aqueous solution (or aqueous dispersion) can be prepared following the procedure of the above drying-micronization method. The spray-drying process can be performed using a known method, thereby giving a powdery pharmaceutical composition in the form of globular particles with the above-mentioned mean particle diameter.

The inhalant suspensions, inhalant solutions, encapsulated inhalants, etc. can also be prepared using the pharmaceutical composition in the form of a powder produced by the drying-micronization method, the spray-drying method and the like, or by using a carrier, additive or excipient and isotopically labeled Pretomanid and/or Delaminid, or isotopically labeled ethionamide and/or prothionamide, or isotopically labeled pyrazinamide, or isotopically labeled isoniazid can be administered via the lungs, according to known preparation methods.

Furthermore, the inhalant comprising the pharmaceutical composition of the invention is preferably used as an aerosol. The aerosol can be prepared, for example, by filling the pharmaceutical composition of the invention and a propellant into an aerosol container. If necessary, dispersants, solvents and the like may be added. The aerosols may be prepared as 2-phase systems, 3-phase systems and diaphragm systems (double containers). The aerosol can be used in any form of a powder, suspension, solution or the like.

Examples of usable propellants include liquefied gas propellants, compressed gases and the like. Usable liquefied gas propellants include, for example, fluorinated hydrocarbons (e.g., CFC substitutes such as HCFC-22, HCFC-123, HFC-134a, HFC-227 and the like), liquefied petroleum, dimethyl ether and the like. Usable compressed gases include, for example, soluble gases (e.g., carbon dioxide, nitric oxide), insoluble gases (e.g., nitrogen) and the like.

The dispersant and solvent may be suitably selected from the additives mentioned above. The aerosol can be prepared, for example, by a known 2-step method comprising the step of preparing the composition of the invention and the step of filling and sealing the composition and propellant into the aerosol container.

One preferred aerosol includes isotopically labeled PA824 and/or OPC67683, propellant fluorinated hydrocarbons such as HFC-134a, HFC-227 or CFC substitutes, solvents including water, ethanol, 2-propanol and the like (water and ethanol are particularly preferable). A weight ratio of water to ethanol in the range of about 0:1 to 10:1 is preferred.

An aerosol of the invention can contain excipients in an amount ranging from about 0.01 to about 10⁴ wt. % (preferably about 0.1 to 10³ wt. %), propellant in an amount of about 10² to 10⁷ wt. % (preferably about 10³ to 10⁶ wt. %), solvent in an amount of about 0 to 10⁶ wt. % (preferably about 10 to 105 wt. %), and dispersant in an amount of 0 to 10³ wt. % (preferably about 0.01 to 10² wt. %), relative to the weight of isotopically labeled Pretomanid and/or Delaminid, or isotopically labeled ethionamide and/or prothionamide, or isotopically labeled pyrazinamide, or isotopically labeled isoniazid which is included in the final composition.

The pharmaceutical compositions of the invention are safe and effective for use in the diagnostic methods according to the present invention. Although the dosage of the composition of the invention may vary depending on the type of active substance administered (isotopically labeled Pretomanid and/or Delaminid, or isotopically labeled ethionamide and/or prothionamide, or isotopically labeled pyrazinamide, or isotopically labeled isoniazid) as well as the nature (size, weight, etc.) of the subject to be diagnosed, the composition is administered in an amount effective for allowing the diagnostically/pharmacologically active substance to be cleaved to measurable cleavage products. For example, the composition is preferably administered such that the active ingredient can be given to a human adult in a dose of about 0.001 to about 100 mg, about 0.01 mg to about 25 mg, about 0.05 mg to about 15 mg, about 0.1 mg to about 10 mg, about 0.5 mg to about 5 mg, about 1 to about 3 mg, and given in a single dose

The form of the pharmaceutical composition of the invention such as a powder, solution, suspension etc. may be suitably selected according to the type of substance to be administered. Dry powder inhalants, inhalant suspensions, inhalant solutions, encapsulated inhalants and similar known forms of inhalants are preferred. Such forms of inhalants can be prepared by filling the pharmaceutical composition of the invention into an appropriate inhaler such as a metered-dose inhaler, dry powder inhaler, atomizer bottle, nebulizer etc. before use. Of the above forms of inhalants, powder inhalants are preferable.

Administration of labeled drug by inhalation using an inhaler is usually preferable. Since the pharmaceutical composition of the invention allows direct local administration into the airways and in particular, directly to pulmonary tissue, the active substance contained therein produces immediate effects. Furthermore, the composition is formulated as an immediate release product so that cleavage and analysis can begin soon after administration.

In certain embodiments, a control (or baseline or reference) ratio of levels of isotope to levels of corresponding non-isotopic atom are determined by measurements of samples taken from the subject before drug administration or taken from a control population of healthy patients or patients who suffer from a Mycobacterium infection. The control (or baseline or reference) ratio is compared to ratios of levels of isotope to levels of corresponding non-isotopic atom measured in a sample obtained from a subject after administration of isotopically-labeled drug. Increased ratios of levels of isotope to levels of corresponding non-isotopic atom are indicative of a drug-responsive Mycobacterium infection, whereas constant or decreasing ratios of levels of isotope to levels of corresponding non-isotopic atom are indicative of the absence of a Mycobacterium infection or of a Mycobacterium infection which is drug resistant.

For example, control levels of ¹⁵N:¹⁴N ratio are determined in samples taken from a subject or control population prior to administration of ¹⁵N-PA824 and/or ¹⁵N-OPC67683. Subsequent to administration of ¹⁵N-PA824 and/or ¹⁵N-OPC67683, levels of ¹⁵N:¹⁴N ratio are measured in a sample obtained from the subject, and a determination of an increasing ¹⁵N:¹⁴N ratio indicates that the subject suffers from Mycobacterium infection which is responsive to treatment with PA824 and/or OPC67683, whereas a decreasing ¹⁵N:¹⁴N ratio indicates either the absence of a Mycobacterium infection or a Mycobacterium infection which is resistant to treatment with PA824 and/or OPC67683.

In the embodiments described in the preceding two paragraphs, both control levels of isotope to levels of corresponding non-isotopic atom and post-drug administration levels of isotope to levels of corresponding non-isotopic atom can be determined at a discrete time point or at several time points. Preferably, ratios of isotope:corresponding non-isotope are measured in a gas sample of the exhaled breath of the subject. (Urine, serum or plasma samples can also be analyzed for nitrite or nitrate levels from nitro cleavage and NO-formation of nitrite and/or nitrate). Gas samples can be obtained in a number of ways, e.g. by having the subject exhale or blow into a tube connected to the measuring instrument. A breath collection bag, a glass vial containing a septum or a nasal cannula can be used. The subject can breath directly into the breath collection bag or through a septum into the glass vial. A nasal cannula could be inserted into a nostril and connected to a measuring instrument. Pulmonary samples can be taken intra-tracheally.

The type of measuring instrument used to detect the product or products depends upon the type of label. Examples of measuring instruments which can be used with nitrogen-15 isotopically labeled nitric oxide (NO.) include, but are not limited to, an infrared spectrometer (see U.S. Pat. No. 5,063,275, which is incorporated by reference herein) and an isotope ratio mass spectrometer. Infrared spectrometers are well known in the art, and have the advantage of being rapid, accurate and sensitive. Preferably, a mass spectrometer gas analyzer or an infrared laser spectrometer is used. For example, if a ¹⁵N isotopically-labelled substrate is used, the ¹⁵N isotopically-labelled cleavage product or products can be detected by using a mass spectrometer or a gas analyzer which is sensitive to ¹⁵N.

One representative test protocol includes the following steps. Isotopically labeled Pretomanid and/or Delaminid, or isotopically labeled ethionamide and/or prothionamide, or isotopically labeled pyrazinamide, or isotopically labeled isoniazid is administered orally or by inhalation to a subject who may suffer from a M. tuberculosis infection. Generally, an effective amount of isotopically-labeled drug ranges from about 0.05 to about 25 mg, about 0.25 to about 10 mg, about 0.5 to about 8 mg, about 0.5 to about 5 mg, about 0.75 to about 3 mg). After an appropriate period of fasting and prior to administration of PA824 and/or OPC67683 (or ethionamide and/or prothionamide, or pyrazinamide), a “control ratio” or baseline ratio of isotopically labeled atom to non-isotopically labeled atom is determined.

Stable Isotope Derivatives of PA824 and OPC67683 can be Used Clinically to Detect Activation and Provide Novel Diagnostic Approaches to Detect TB and PA824/OPC67683 Resistance and/or Sensitivity.

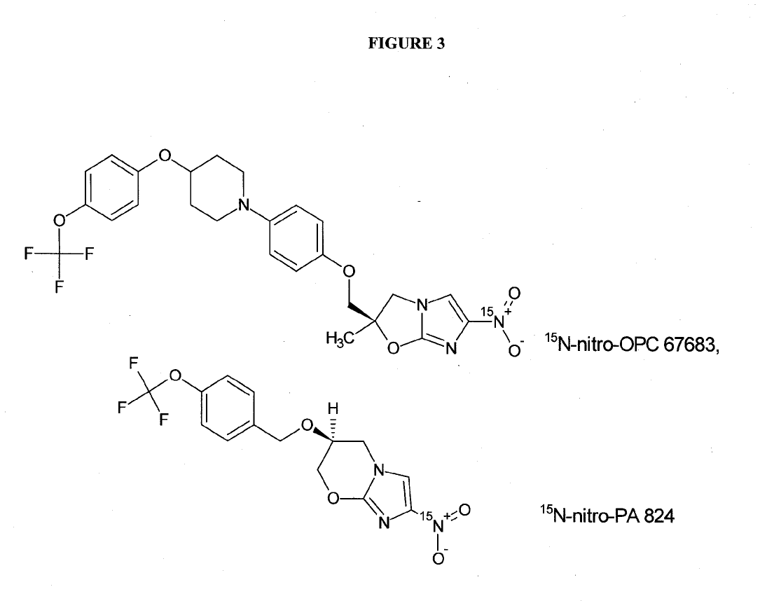

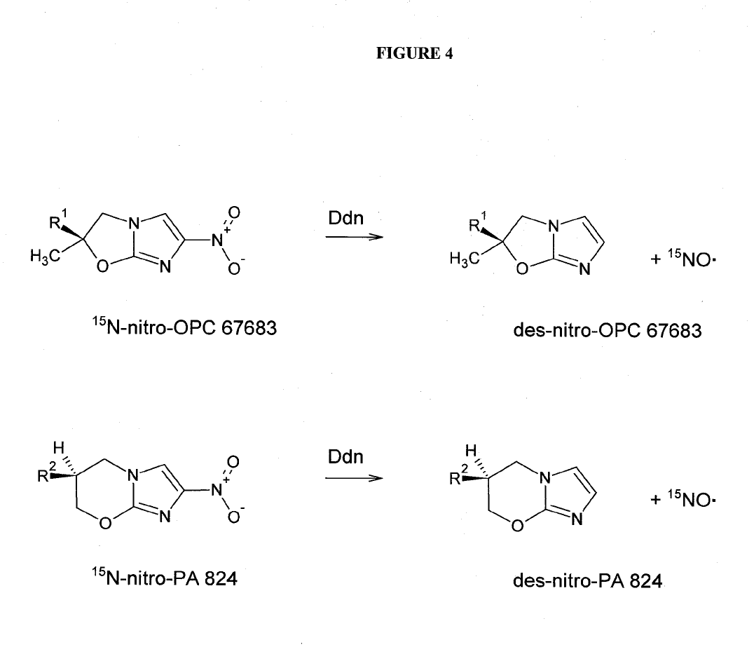

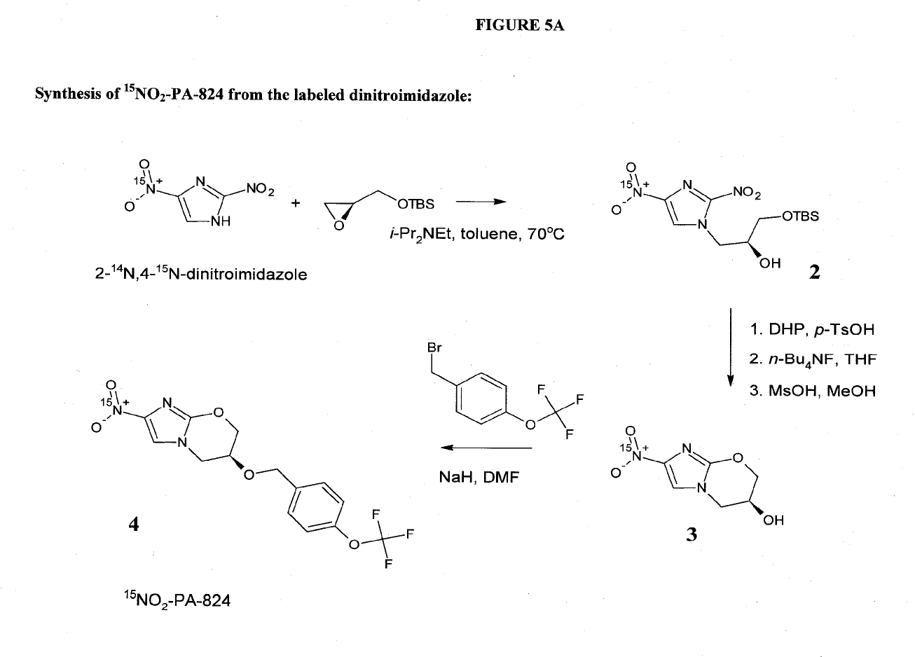

By labeling PS824 or OPC67682 in the nitro substituent nitrogen with ¹⁵N, we have produced PA824 and OPC 67683 labeled with ¹⁵N at the nitro group (FIG. 3). Upon activation of labeled PA824 or OPC67683, ¹⁵NO• (and its nitrate and nitrite metabolites) is produced. The detection of this ¹⁵NO• in breath by mass spectrometry or other spectroscopy affords a technique to detect PA824/OPC67683 activation, and thus enable detection of TB, and its sensitivity or resistance to PA824/OPC67683 based upon the extent of conversion. This could also be performed on other fluids such as blood, saliva or urine either directly or after metabolism of the ¹⁵NO• to its nitrate and nitrate metabolites by normal human metabolism.

In a preferred embodiment, ¹⁵N-labeled (at the NO₂ substituent) PA824/OPC67683 is administered by inhalation (e.g. using a nebulizer, dry powder inhaler, or by instillation), and the levels of ¹⁵NO• are measured in breath samples taken both before labeled PA824/OPC67683 administration and at time points thereafter. Increases in ¹⁵NO• shortly after administration indicate PA824/OPC67683 activation by lung Mycobacteria, and are predictive of PA824/OPC67683 sensitivity. Thus, ¹⁵N-labeled PA824/OPC67683 and its conversion to ¹⁵NO• by M. tuberculosis, represents a viable approach for determining Myccobacterial sensitivity and/or resistance to PA824 and/or OPC67683.

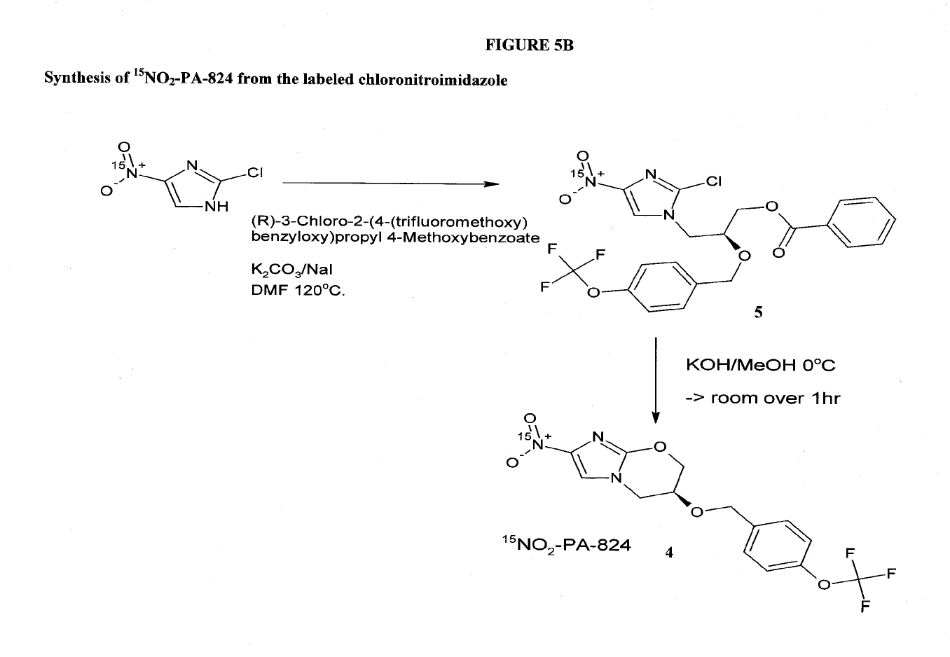

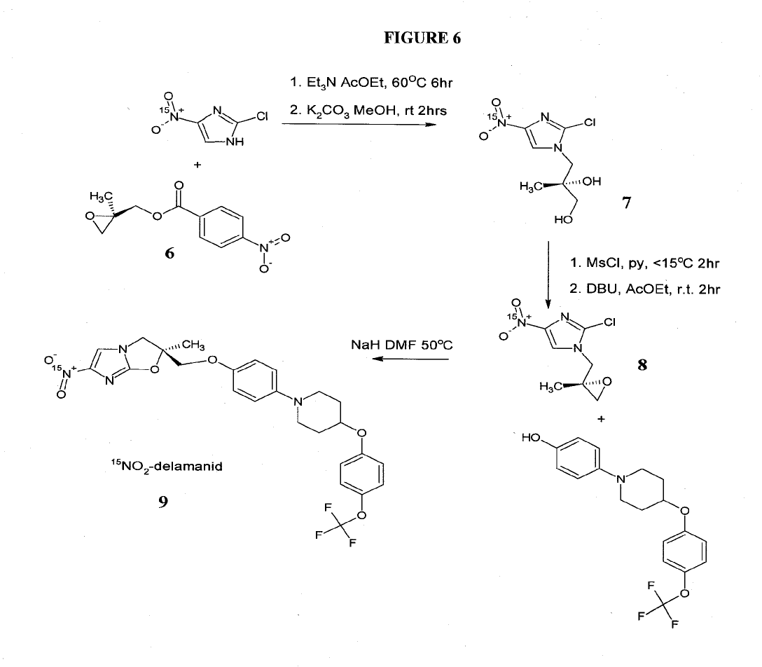

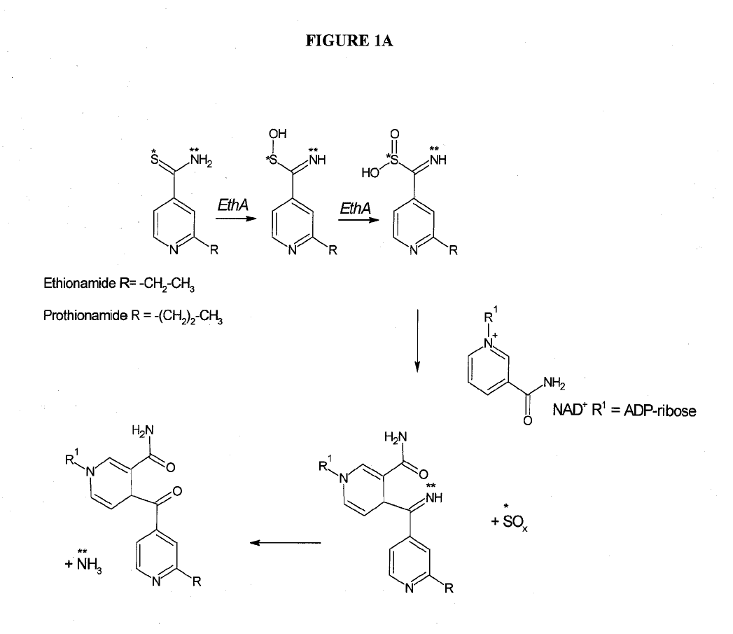

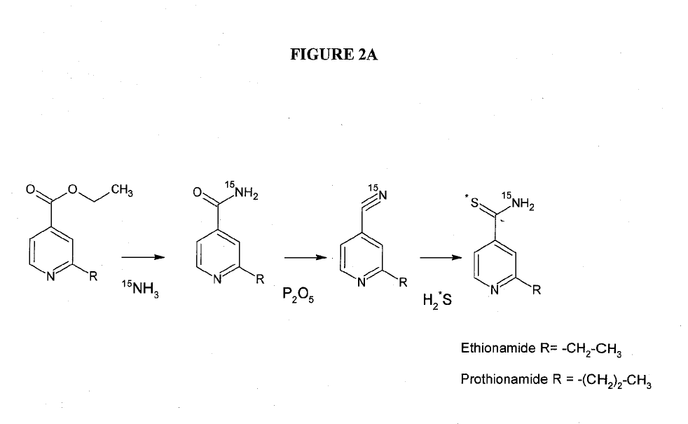

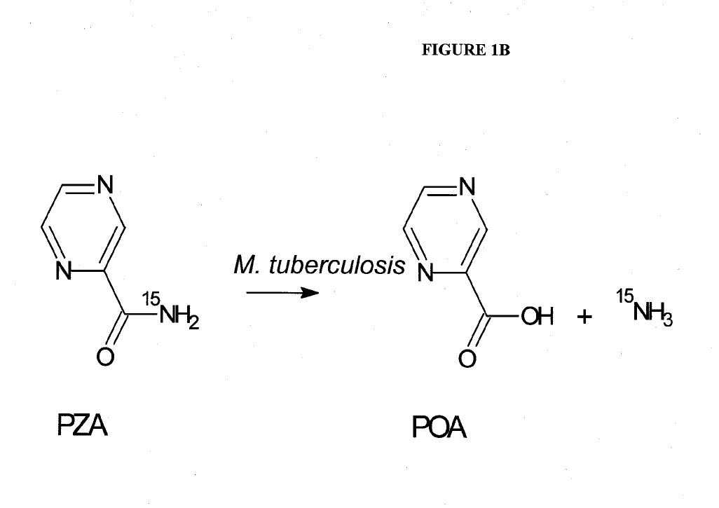

We labeled PA824 and OPC67683 with ¹⁵N at the nitro group (FIG. 5). Upon activation of ¹⁵N-labeled PA824 and/or OPC67683, ¹⁵NO• is produced. The detection of ¹⁵NO• in breath by mass spectrometry or other spectroscopy affords a technique to detect PA824 and/or OPC67683 activation, and enables detection of TB, and a determination of its sensitivity or resistance to PA824 and/or OPC67683 based upon the extent of isotopically-labeled drug conversion. Blood, saliva or urine levels of ¹⁵NO• can be measured similarly.

Stable Isotope Derivatives of Ethionamide and/or Prothionamide can be Used Clinically to Detect Activation and Provide New Diagnostic Approaches to Detect TB and Ethionamide/Prothionamide Resistance or Sensitivity.

By labeling ethionamide and/or prothionamide in the thioamide nitrogen or sulfur with ¹⁵N or isotopic sulfur (³³S, ³⁴S or more often ³⁶S) following the approach of Liberman, U.S. Pat. No. 2,901,488, we have produced ethionamide and prothionamide labeled with ¹⁵N or isotopic sulfur at the thioamide group (FIG. 1A or 2A). Upon activation of this labeled ethionamide/prothionamide, ¹⁵NH₃, urea, or an isotopically labeled sulfur (³³S, ³⁴S or more often ³⁶S) metabolite is produced. The detection of this ¹⁵NH₃ or sulfur metabolite in breath or in urine, plasma, saliva or sputum, by mass spectrometry or other spectroscopy affords a technique to detect ethionamide/prothionamide activation, and thus enable detection of TB, and its sensitivity or resistance to ethionamide/prothionamide based upon the extent of conversion. Our methods can also be performed on other fluids such as blood, saliva, urine or sputum, either directly or after metabolism of the ¹⁵NH₃ to labeled urea or by measuring sulfur metabolites of normal human metabolism. Metabolites of ¹⁵NH₃ other than urea may also be measured.

One embodiment of the present invention is directed to a composition comprising ¹⁵N- and/or ³³S, ³⁴S or ³⁶S-ethionamide/prothionamide or related compounds.

In an alternative embodiment, the invention is directed to the compounds ¹⁵N-ethionamide/prothionamide and/or ³³S, ³⁴S or ³⁶S-ethionamide/prothionamide which are represented by the following chemical structure:

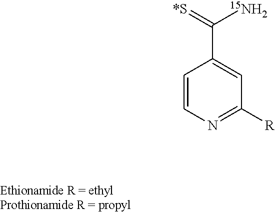

In the above compound, S and/or NH₂ are isotopically labeled.

In an alternative embodiment, the present invention is directed to a method for synthesizing ¹⁵N-ethionamide/propionamde and/or isotopically labeled (³³S, ³⁴S or ³⁶S)-ethionamide/prothionamide.

In still further embodiments, the present invention is directed to a method for detecting ethionamide/prothionamide metabolism by Mycobacteria in a patient, wherein the identification of isotopically labeled ammonia or urea (from the metabolism of ¹⁵N-isotopically labeled ethionamide/prothionamide) and/or isotopically labeled (³³S, ³⁴S or ³⁶S) sulfur metabolites (such as various SOₓ metabolites) evidences that the mycobacterium is ethionamide sensitive and if little or no isotopically labeled sulfate metabolites, ammonia or urea is measured, this evidences that the Mycobacterium is resistant to ethionamide/prothionamide. It is noted that one preferred first or subsequent step is to identify that the patient has a Mycobacterium, including a tuberculosis infection by alternative means if the present method evidences resistance of the bacterial infection to ethionamide/prothionamide pursuant to the present invention. The present method can be performed in the absence or presence of drugs which increase or boost the activity of ethionamide/prothionamide.

In still further embodiments, the present invention is directed to the detection of ¹⁵NH₃ in breath, blood, saliva, urine and sputum in a patient with a Mycobacterium infection who has been administered ¹⁵N-labeled ethionamide/prothionamide. In still further embodiments, the present invention is directed to the detection of ¹⁵N-urea in blood, saliva, urine or sputum in a patient with a Mycobacterium infection who has been administered ¹⁵N-labeled ethionamide/prothionamide. In still further embodiments, the present invention is directed to the detection of ³³S, ³⁴S or ³⁶S-labeled (SOₓ) metabolites in blood, saliva, urine and/or sputum.

In still additional embodiments, the present invention is directed to the use of a breath test with inhaled ¹⁵N-ethionamide/prothionamide for rapid detection of ¹⁵NH₃. Evidence of isotopically labeled ammonia in the breath of a patient with a Mycobacterium infection is evidence that the infection is sensitive to ethionamide/prothionamide and the absence of or substantially reduced labeled ammonia in the breath of the patient is evidence that the Mycobacterium infection is resistant to ethionamide/prothionamide.

In cases where the bacteria appears to be resistant to ethionamide/prothionamide and other symptoms evidence that the patient has a Mycobacterium, including a tuberculosis infection, other tests should be used to determine/confirm the existence of infection in the patient. This can include other breath tests and/or culturing lung tissue and/or sputum from the patient to determine the existence of the bacteria.

In one embodiment of the present invention, the labeled NH₃ is collected by absorption to an ammonia absorbing matrix, and eluted prior to or during analysis. In still a further embodiment, the diagnostic method is multiplexed with other isotope labeled TB drugs so that multiple drug sensitivities can be determined rapidly in a single diagnostic test.

By labeling ethionamide/prothionamide in the thioamide nitrogen with ¹⁵N by reactions detailed in FIG. 2A we produce ethionamide/prothionamide labeled with ¹⁵N at the thioamide group (FIG. 1A). Upon activation of this labeled ethionamide/prothionamide, ¹⁵NH₃ is produced. The detection of this ¹⁵NH₃ in breath by mass spectrometry or other spectroscopy affords a technique to detect ethionamide/prothionamide activation, and thus enable detection of TB, and its sensitivity or resistance to ethionamide/prothionamide based upon the extent of conversion. This could also be performed on other fluids such as blood, saliva or urine either directly or after metabolism of the ¹⁵NH₃ to labeled urea by normal human metabolism. Alternatively or in addition, we can label the ethionamide/prothionamide with isotopically labeled sulfur in the thioamide moiety as indicated in FIG. 2A. Detection of labeled-SOₓ metabolites in blood, urine, saliva, sputum or other samples can similarly be used.

Levels of ¹⁵N-ammonia and other ¹⁵N, ³³S, ³⁴S or ³⁶S labeled metabolites (e.g. SOₓ metabolites) can be measured in the blood, urine, saliva or sputum.

Diagnostic tests can also be multiplexed with other isotopically labeled prodrugs such as isoniazid (producing ¹⁵NO or ¹⁵N₂), pyrazinamide (producing ¹⁵NH₃), Delamanid and PA-824 (producing ¹⁵NO and other nitrogen oxides), so that more than one drug sensitivity or resistance can be measured by measuring different levels. For example, isoniazid activation by KatG and thus sensitivity could be determined by measuring breath ¹⁵N₂ after administration of ¹⁵N₂-hydrazyl isoniazid, and this could be performed concurrently with administration of labeled ethionamide as described here in which products such as ¹⁵NH₃ could be detected after administration.

Amide-¹⁵N-Pyrazinamide (¹⁵N-PZA) and its Conversion to ¹⁵NH₃ by M. tuberculosis.

Stable isotope derivatives of PZA can be used clinically to detect PZA activation and provide new diagnostic approaches to detect M. tuberculosis resistance and/or sensitivity to PZA treatment.

¹⁵N-isotopically labeled pyrazinamide yields the M. tuberculosis metabolic/cleavage products ¹⁵NH₃ and ¹⁵N-urea. In the case of ¹³C or ¹⁷O/¹⁸O-labeled pyrazinamide, the metabolic/cleavage products are ¹³C or ¹⁷O/¹⁸O-pyrazinoic acid.

By labeling PZA in the amide nitrogen with ¹⁵N by reaction of ¹⁵N₂-urea with pyrazine 2, 3 dicarboxylic acid, the inventors have produced PZA labeled with ¹⁵N at the amide group (FIG. 1B). Upon activation of this labeled PZA ¹⁵NH₃ is produced. The detection of this ¹⁵NH₃ in breath by mass spectrometry or other spectroscopy affords a technique to detect PZA activation, and thus enable detection of TB, and its sensitivity or resistance to PZA based upon the extent of conversion. This could also be performed on other fluids such as blood, saliva or urine either directly or after metabolism of the ¹⁵NH₃ to labeled urea by normal human metabolism. Alternatively, we can label the PZA with ¹³C, such as by labeled o-phenylene diamine to make labeled quinoxaline which is then used to make ¹³C-labeled PZA. Detection of ¹³C-POA in blood or other samples can similarly be used.

In a preferred embodiment, ¹⁵N-amide labeled PZA is administered by inhalation (e.g. using a nebulizer, dry powder inhaler, or by instillation), and the levels of ¹⁵NH₃ are measured in a subject's breath both before labeled PZA administration, and at time points thereafter. Increases in ¹⁵NH₃ shortly after indicate PZA activation by lung mycobacteria, and are predictive of PZA sensitivity. Later increases in ¹⁵NH₃ may be due to either mycobacteria or other pathways, such as hepatic microsomal deamidase by the host that leads to peak POA levels at ˜6 hours after oral dosage FIG. 1B.

¹⁵N-amide-pyrazinamide (¹⁵N-PZA) can be synthesized using a modified version of the syntheses described in U.S. Pat. No. 2,705,714. Briefly, pyrazine 2, 3-dicarboxylic acid is heated with ¹⁵N₂-urea; ¹⁵N-PZA is collected by sublimation and recrystallized.

Thus, in one embodiment, the present invention is directed to a composition comprising ¹⁵N-pyrazinamide and/or ¹³C-pyrazinamide or related compounds. In an alternative embodiment, the invention is directed to the compounds ¹⁵N-pyrazinamide and/or ¹³C-pyrazinamide which are represented by the following chemical structures:

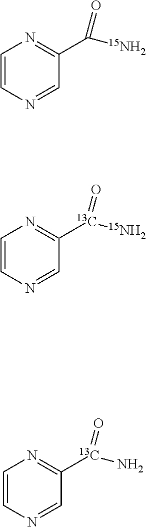 [SMILES: [15NH2]C(=O)c1cnccn1 ; [15NH2][13C](=O)c1cnccn1 ; N[13C](=O)c1cnccn1]

In additional embodiment, one or more of the oxygen groups in the amide of pyrazinamide, including any one or more of the above compounds may be substituted with ¹⁷O or ¹⁸O.

In an alternative embodiment, the present invention is directed to a method for synthesizing ¹⁵N-pyrazinamide and/or ¹³C-pyrazinamide.

In still further embodiments, the present invention is directed to a method for detecting PZA metabolism by Mycobacteria in a patient, wherein the identification of isotopically labeled pyrazinoic acid (by virtual of identifying isotopically labeled pyrazinoic acid containing ¹³C and/or ¹⁷O/¹⁸O from the metabolized isotopically labeled pyrazinamide) or isotopically labeled ammonia or urea (from the metabolism of isotopically labeled pyrazinamide) evidences that the mycobacterium is pyrazinamide sensitive and if little or no isotopically labeled pyrazinoic acid, ammonia or urea is measured, this evidences that the Mycobactirium is resistant to pyrazinamide.

In still further embodiments, the present invention is directed to the detection of ¹⁵NH₃ in breath, blood, saliva, urine in a patient with a Mycobacterium infection who has been administered ¹⁵N-labeled pyrazinamide. In still further embodiments, the present invention is directed to the detection of ¹⁵N-urea in blood, saliva, urine in a patient with a Mycobacterium infection who has been administered ¹⁵N-labeled pyrazinamide. In still further embodiments, the present invention is directed to the detection of ¹³C-labeled pyrazinoic acid (POA) in blood, saliva, urine.

In still additional embodiments, the present invention is directed to the use of a breath test with inhaled ¹⁵N-pyrazinamide for rapid detection of ¹⁵NH₃. Evidence of isotopicallly labeled ammonia in the breath of a patient with a Mycobacteriium infection is evidence that the infection is sensitive to pyrazinamide and the absence of or substantially reduced labeled ammonia in the breath of the patient is evidence that the Mycobacterium infection is resistant to pyrazinamide.

By labeling PZA in the amide nitrogen with ¹⁵N by reaction of ¹⁵N₂-urea with pyrazine 2,3 dicarboxylic acid, we produce PZA labeled with ¹⁵N at the amide group (FIG. 1B). Upon activation of this labeled PZA ¹⁵NH₃ is produced. The detection of this ¹⁵NH₃ in breath by mass spectrometry or other spectroscopy affords a technique to detect PZA activation, and thus enable detection of TB, and its sensitivity or resistance to PZA based upon the extent of conversion. This could also be performed on other fluids such as blood, saliva or urine either directly or after metabolism of the ¹⁵NH₃ to labeled urea by normal human metabolism. Alternatively, we can label the PZA with ¹³C, such as by labeled o-phenylene diamine to make labeled quinoxaline which is then used to make ¹³C-labeled PZA. Detection of ¹³C-POA in blood or other samples can similarly be used.

In a preferred embodiment, ¹⁵N-amide labeled PZA is given by an inhaled route (nebulization, dry powder inhaler, instillation), and the levels of ¹⁵NH₃ measured in breath both before labeled PZA administration, and at time points thereafter. Increases in ¹⁵NH₃ shortly after indicate PZA activation by lung mycobacteria, and are predictive of PZA sensitivity. Later increases in ¹⁵NH₃ may be due to either mycobacteria or other pathways, such as hepatic microsomal deamidase by the host that leads to peak POA levels at ˜6 hours after oral dosage. Interaction between allopurinol and pyrazinamide Lacroix et al. Eur Resp. J. 1988 1 807-11.

¹⁵NH₃ and/or ¹⁵N-urea are detectable if ¹⁵N-pyrazinamide (PZA) was administered to the subject, and/or ¹³C-pyrazinoic acid is detectable if ¹³C-pyrazinamide was administered to the subject, and/or ¹⁷O-pyrazinoic acid or ¹⁸O-pyrazinoic acid are detectable if an oxygen isotope-labeled pyrazinamide was administered to the subject

The invention is illustrated further in the following non-limiting examples.

### Example 1

### Synthesis of ¹⁵N-nitro-PA824 and ¹⁵N-nitro-delamanid

Modifications of either route described by Marisini, et al ¹⁹ (which is incorporated by reference in its entirety herein) can be used starting from ¹⁵N-dinitroimidazole or ¹⁵N-chloro-nitro-imidazole. The chemical synthetic scheme for these starting materials are set forth in FIGS. 5A and 5B.

### ¹⁵N-nitro-PA824

In FIG. 5A, starting from ¹⁵N-dinitroimidazole, this material is reacted with a silyl-protected cyclopropylmethyl alcohol in tertiary amine at elevated temperature to provide the dinitroimidazolyl substituted silyl-protected propane diol derivative 2 which is subsequently cyclized to the bicyclic imidazole pyranyl intermediate 3 in DHP/acid (preferably toluene sulphonic acid), followed by silyl deprotection (fluoride/solvent, preferably n-Bu₄NF, THF) and cyclization (acid/solvent, preferably MsOH/MeOH). The imidazole pyran bicyclic intermediate is then reacted with p-bromomethyl-trifluoromethoxy benzene in the presence of strong base and solvent (e.g. NaH/DMF) to produce ¹⁵NO₂—PA-824.

¹⁵NO₂—PA-824 (4) is also synthesized from ¹⁵N-chloro-nitro-imidazole (FIG. 5B) by reacting that starting material with (R)-3-Chloro-2-(4-(trifluoromethoxy)benzyloxy)propyl 4-Methoxy benzoate in weak based and iodide anion in solvent at elevated temperature (e.g., potassium carbon+ate/sodium iodide in DMF at 120° C.) to provide the condensed intermediate 5 which is cyclized in base and solvent (e.g. potassium hydroxide/methanol at 0° C. followed by further reaction at room temperature for an hour or more) to provide ¹⁵NO₂-PA-824 (4).

¹⁵N-nitro-delamanid

¹⁵N-nitro-delamanid (9) is synthesized from ¹⁵N-chloro-nitro-imidazole and p-nitrophenyl methyl glycidyl ester (6) in triethylamine/solvent (ethylacetate) followed by weak base (e.g. potassium carbonate in methanol at room temperature) to form the chloro-nitro-imidazole methyl propyl diol (7), which is reacted with MsCl in pyridine at slightly lower than room temperature, followed by DBU in ethyl acetate at room temperature for 2 hours to form the methyl glycidyl ¹⁵N-chloro-nitro-imidazole compound (8). Compound 8 is reacted with the p-trifluoromethoxyphenoxy piperidinyl phenol reactant in strong base (NaH/solvent) at elevated temperature (e.g. 50° C.) to produce ¹⁵N-nitro-delamanid (9).

### Phenotypic Analysis of Resistance

Although studies of clinical resistance to PA824 (Pretomanid) and/or OPC67683 (Delaminid) are only just beginning, it is known that in vitro resistant strains are mutants with lowered drug activation.^{2,3} ../../Documents and Settings/R Alghieri/Local Settings/AppData/Local/Microsoft/Windows/Temporary Internet Files/OLK4BEE/N12-243Prov.delamanid and PA824 breath test disclosure 2-2014.docx—_ENREF_3# ENREF_3 It appears that PA824 (Pretomanid)/OPC67683 (Delaminid) resistance can be associated with mutations in many different genes ⁶ including:

a) Ddn, the enzyme that directly activates PA824/Delamanid

b) Fgd1, the enzyme that recycles the F₄₂₀ cofactor used by Ddn

c) FbiA, FbiB or FbiC, genes that are involved in the synthesis of F₄₂₀ cofactor Since neither Ddn or Fdg1 has been found to be essential 1⁶ nor is either FbiA⁷ or FbiB⁸ essential. Therefore, it is highly unlikely that selection pressures to maintain these activities will overcome pressures for selection of PA824/delamanid resistance, and so a very wide range of loss of function mutations in these different genes will occur that will be very difficult to collate, understand and rank importance of in clinical use of these drugs. Furthermore, since the active metabolite NO• reacts non-specifically with a wide range of cellular targets, it is unlikely that mutation in any one target would be selected for to produce resistance either.

An analogy is another anti-TB prodrug pyrazinamide, in which all known resistance maps to mutations to just one activating enzyme PncA. However, we do not yet know what the patterns of genetic mutations are in the activation pathway that will drive resistance development, and are thus unable to use rapid molecular techniques such as are used for rifampin resistance in GenExpert MTB/RIF assay(http://www.tbdreamdb.com/PZA_Rv2043c_AllMutations.html). The situation for PA824/Delamanid is likely to be much more complicated than PZA due to the potential for mutations in not one, but 5 key genes.

Thus, early usage of PA824 and OPC 67683 will be guided by phenotypic drug resistance assays (such as the Bactec MGIT) that require sputum collection (which may be difficult or impossible) and that use growth as the readout entailing times from test to result of a few weeks to a few months. This delay will result in many negative outcomes including ineffective treatment of already drug-resistant TB, more rapid development of resistance to PA824/OPC 67683, and more transmission of resistant strains.

There is therefore a major need for a much more rapid phenotypic assay of PA824/OPC67683 resistance, both to allow the optimal use of these new weapons against TB, and to ultimately allow molecular diagnostics by rapidly associating drug activation mutations with their phenotypic resistance. By rapidly developing and implementing such a rapid phenotypic diagnostic, during the clinical roll-out of these drugs rather than years afterwards, we will be able to significantly extend their duration of impact and clinical lifespan.

Isotopic Breath Test of Ddn Activity-Rapid Phenotype without Culture or Sputum.

We have been active in developing a number of phenotype diagnostics for bacterial lung infection based upon stable isotope labeled substrates and detection of labeled volatiles from their bacterial metabolism. We here delineate how novel stable isotope derivatives of PA 824 or OPC 67683 can be used clinically to detect Ddn activation and provide new diagnostic approaches to detect resistance to these drugs. By labeling the nitro group of either compound with enriched ¹⁵N we can produce the new chemical entities ¹⁵N-nitro-PA824 and ¹⁵N-nitro-OPC67683 shown in FIG. 3. Upon delivery of these compounds to drug sensitive-TB in patients, either through inhalation, oral or intravenous delivery, active Ddn will convert the drug to the des-nitro form and release labeled ¹⁵NO•. Thus an increase in ¹⁵NO• in exhaled breath after delivery of ¹⁵N-nitro-PA824 or ¹⁵N-nitro-OPC67683 would be diagnostic of active Ddn, and hence the presence of drug-sensitive TB.

As shown in FIG. 2, we established the activation of PA824 and OPC67683 to NO• by Ddn. FIG. 4 shows the Ddn mediated production of ¹⁵NO• from ¹⁵N-nitro-OPC67683 and ¹⁵N-nitro-PA824.

Alternatively, any of the known degradation products of ¹⁵NO•, such as ¹⁵N-nitrate or nitrite (¹⁵NO₃⁻ or ¹⁵NO₂⁻) can be measured in a range of samples including but not limited to such as blood, urine, saliva or exhaled breath condensate.

### Example 2

### Synthesis of ¹⁵N-thioamide-ethionamide/prothionamide

From the appropriately substituted ethylisonicotinate analog, synthesis with labeled nitrogen or sulfur is conducted as previously described by Liberman, U.S. Pat. No. 2,901,488. See FIG. 2A.

### Example 3

### Synthesis of ¹⁵N-amide-pyrazinamide (¹⁵N-PZA)

Synthesis is based upon modifications of the techniques described in U.S. Pat. No. 2,705,714. Pyrazine 2, 3-dicarboxylic acid is heated with ¹⁵N₂-urea. ¹⁵N-PZA is collected by sublimation and is re-crystallized.

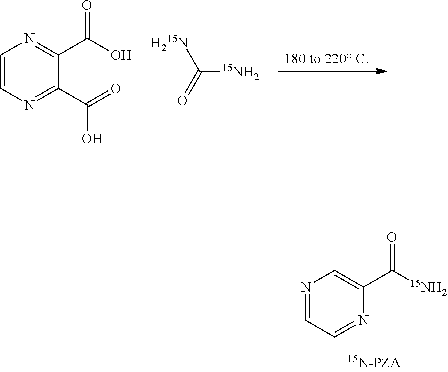

### REFERENCES

1 Stover, C. K. et al. A small-molecule nitroimidazopyran drug candidate for the treatment of tuberculosis. Nature 405, 962-966, doi:10.1038/35016103 (2000).
2 Matsumoto, M. et al. OPC-67683, a nitro-dihydro-imidazooxazole derivative with promising action against tuberculosis in vitro and in mice. PLoS medicine 3, e466, doi:10.1371/journal.pmed.0030466 (2006).
3 Gler, M. T. et al. Delamanid for multidrug-resistant pulmonary tuberculosis. The New England journal of medicine 366, 2151-2160, doi:10.1056/NEJMoa 112433 (2012).
4 Gurumurthy, M. et al. Substrate specificity of the deazaflavin-dependent nitroreductase from Mycobacterium tuberculosis responsible for the bioreductive activation of bicyclic nitroimidazoles. Febs J 279, 113-125, doi:10.1111/j.1742-4658.2011.08404.x (2012).
5 Singh, R. et al. PA-824 kills nonreplicating Mycobacterium tuberculosis by intracellular NO release. Science 322, 1392-1395, doi:10.1126/science.1164571 (2008).
6 Feuerriegel, S. et al. Impact of Fgd1 and ddn diversity in Mycobacterium tuberculosis complex on in vitro susceptibility to PA-824. Antimicrob Agents Chemother 55, 5718-5722, doi:10.1128/AAC.05500-11 (2011).
7 Sassetti, C. M., Boyd, D. H. & Rubin, E. J. Genes required for mycobacterial growth defined by high density mutagenesis. Mol Microbiol 48, 77-84 (2003).
8 Griffin, J. E. et al. High-resolution phenotypic profiling defines genes essential for mycobacterial growth and cholesterol catabolism. PLoS pathogens 7, e1002251, doi:10.1371/journal.ppat.1002251 (2011).
9 Marsini, M. A., Reider, P. J. & Sorensen, E. J. A concise and convergent synthesis of PA-824. The Journal of organic chemistry 75, 7479-7482, doi:10.1021/jo1015807 (2010).
1A DeBarber, A. E., Mdluli, K., Bosman, M., Bekker, L.-G. & Barry, C. E. Ethionamide activation and sensitivity in multidrug-resistant Mycobacterium tuberculosis. Proceedings of the national academy of sciences 97, 9677-9682 (2000).
2A Qian, L. & Ortiz de Montellano, P. R. Oxidative activation of thiacetazone by the Mycobacterium tuberculosis flavin monooxygenase EtaA and human FMO1 and FMO3. Chem Res Toxicol 19, 443-449, doi:10.1021/tx050328b (2006).
3A Vannelli, T. A., Dykman, A. & Ortiz de Montellano, P. R. The antituberculosis drug ethionamide is activated by a flavoprotein monooxygenase. J Biol Chem 277, 12824-12829, doi:10.1074/jbc.M110751200 (2002).
4A Wang, F. et al. Mechanism of thioamide drug action against tuberculosis and leprosy. Journal of Experimental Medicine 204, 73-78, doi:Doi 10.1084/Jem.20062100 (2007).
5A Baulard, A. R. et al. Activation of the pro-drug ethionamide is regulated in mycobacteria. J Biol Chem 275, 28326-28331 (2000).
6A Flipo, M. et al. Discovery of novel N-phenylphenoxyacetamide derivatives as EthR inhibitors and ethionamide boosters by combining high-throughput screening and synthesis. Journal of medicinal chemistry 55, 6391-6402 (2012).
7A Flipo, M. et al. Ethionamide boosters: synthesis, biological activity, and structure-activity relationships of a series of 1, 2, 4-oxadiazole EthR inhibitors. Journal of medicinal chemistry 54, 2994-3010 (2011).
8A Flipo, M. et al. Ethionamide boosters. 2. Combining bioisosteric replacement and structure-based drug design to solve pharmacokinetic issues in a series of potent 1, 2, 4-oxadiazole EthR inhibitors. Journal of medicinal chemistry 55, 68-83 (2011).
9A Morlock, G. P., Metchock, B., Sikes, D., Crawford, J. T. & Cooksey, R. C. ethA, inhA, and katG loci of ethionamide-resistant clinical Mycobacterium tuberculosis isolates. Antimicrobial agents and chemotherapy 47, 3799-3805 (2003).
10A Brossier, F., Veziris, N., Truffot-Pernot, C., Jarlier, V. & Sougakoff, W. Molecular investigation of resistance to the antituberculous drug ethionamide in multidrug-resistant clinical isolates of Mycobacterium tuberculosis. Antimicrobial agents and chemotherapy 55, 355-360 (2011).
11A Francois, A. A., Nishida, C. R., de Montellano, P. R., Phillips, I. R. & Shephard, E. A. Human flavin-containing monooxygenase 2.1 catalyzes oxygenation of the antitubercular drugs thiacetazone and ethionamide. Drug metabolism and disposition: the biological fate of chemicals 37, 178-186, doi: 10.1124/dmd.108.024158 (2009).
12A Marsini, M. A., Reider, P. J. & Sorensen, E. J. A concise and convergent synthesis of PA-824. The Journal of organic chemistry 75, 7479-7482, doi:10.1021/jo1015807 (2010).

1. A method of identifying the sensitivity and resistance to therapeutic drug regimens in a subject who suffers from, or who is suspected of suffering from, a Mycobacterium infection, the method comprising:
(a) administering ¹⁵N-nitro-PA824 (¹⁵N-nitro-Pretomanid) and/or ¹⁵N-nitro-OPC67683 (¹⁵N-nitro-Delaminid) to the subject and thereafter measuring levels in a subject-derived sample of one or more markers selected from the group consisting of ¹⁵NO or ¹⁵NO degradation products ¹⁵N-nitrate or nitrite (¹⁵NO₃⁻ or ¹⁵NO₂⁻), wherein detectable levels in the sample of ¹⁵NO• or ¹⁵NO₃⁻ or ¹⁵NO₂ indicate that the subject suffers a Mycobacterium infection which is susceptible to treatment with PA824 (Pretomanid) and/or OPC67683 (Delaminid), and wherein the absence of detectable levels of ¹⁵NO• or ¹⁵NO indicates either that the subject does not suffer from a Mycobacterium infection or suffers from a Mycobacterium infection which is resistant to treatment with PA824 (Pretomanid) and/or OPC67683 (Delaminid); and/or
(b) administering (1) ¹⁵N-ethionamide, or a sulfur isotope-labeled ethionamide selected from the group consisting of ³³S-ethionamide, ³⁴S-ethionamide or ³⁶S-ethionamide and/or (2) ¹⁵N-prothionamide or a sulfur isotope-labeled prothionamide selected from the group consisting of ³³S-prothionamide, ³⁴S-prothionamide or ³⁶S-prothionamide to the subject and thereafter measuring levels in a subject-derived sample of (i) ¹⁵NH₃ if ¹⁵N-ethionamide or ¹⁵N-prothionamide was administered to the subject, and/or (ii) sulfur oxides of the formula SOₓ, where x is an integer from 2 to 4, if a sulfur isotope-labeled ethionamide or sulfur isotope-labeled prothionamide was administered to the subject, wherein detectable levels in the sample of ¹⁵NH₃ or sulfur oxides indicate that the subject suffers a Mycobacterium infection which is susceptible to treatment with ethionamide and/or prothionamide, and wherein the absence of detectable levels of ¹⁵NH₃ or sulfur oxides indicate either that the subject does not suffer from a Mycobacterium infection or suffers from a Mycobacterium infection which is resistant to treatment with ethionamide and/or prothionamide; and/or
(c) administering ¹⁵N-pyrazinamide (PZA) and/or ¹³C-pyrazinamide and/or an oxygen isotope-labeled pyrazinamide selected from the group consisting of ¹⁷O-pyrazinamide and ¹⁸O-pyrazinamide to the subject and thereafter measuring levels in a subject-derived sample of ¹⁵NH₃ if ¹⁵N-pyrazinamide (PZA) was administered to the subject, and/or ¹³C-pyrazinoic acid if ¹³C-pyrazinamide was administered to the subject, and/or ¹⁷O-pyrazinoic acid or ¹⁸O-pyrazinoic acid if an oxygen isotope-labeled pyrazinamide was administered to the subject, wherein detectable levels in the sample of ¹⁵NH₃, ¹³C-pyrazinoic acid, and/or ¹⁷O-pyrazinoic acid or ¹⁸O-pyrazinoic acid indicate that the subject suffers from a Mycobacterium infection which is susceptible to treatment with pyrazinamide, and wherein the absence of detectable levels of ¹⁵NH₃, ¹³C-pyrazinoic acid, and/or ¹⁷O-pyrazinoic acid or ¹⁸O-pyrazinoic acid indicate either that the subject does not suffer from a Mycobacterium infection or suffers from a Mycobacterium infection which is resistant to treatment with pyrazinamide; and/or
(d) administering ¹⁵N-isoniazid to the subject and thereafter measuring levels in a subject-derived sample of ¹⁵NO and/or ¹⁵N₂, wherein detectable levels in the sample of ¹⁵NO and/or ¹⁵N₂ indicate that the subject suffers from a Mycobacterium infection which is susceptible to treatment with isoniazid, and wherein the absence of detectable levels of ¹⁵NO and/or ¹⁵N₂ indicate either that the subject does not suffer from a Mycobacterium infection or suffers from a Mycobacterium infection which is resistant to treatment with pyrazinamide.

2. The method of claim 1, wherein the Mycobacterium infection is selected from the group consisting of infections caused by members of the Mycobacterium tuberculosis complex, the Mycobacterium avium complex, the Mycobacterium gordonae clade, the Mycobacterium kansasii clade, the Mycobacterium nonchromogenicum/terrae clade, the Mycolactone-producing mycobacteria, the Mycobacterium simiae clade, the Mycobacterium chelonae clade, the Mycobacterium fortuitum clade, the Mycobacterium parafortuitum clade and the Mycobacterium vaccae clade.

3. The method of claim 1, wherein the Mycobacterium infection is a Mycobacterium tuberculosis infection.

4. The method of claim 1, wherein the Mycobacterium infection is Mycobacterium avium complex (MAC) and Mycobacterium scrofulaceum.

5. The method of claim 1, wherein the sample is a breath, blood, saliva, urine and/or sputum sample.

6. The method of claim 1, wherein more than one sample is taken, an isotopic cleavage product or metabolite plasma concentration-time curve is generated, and the area under the curve (AUC) is calculated to determine the extent of Mycobacterium infection resistance to PA824 (Pretomanid), OPC67683 (Delaminid), ethionamide, prothionamide, pyrazinamide (PZA) or isoniazid treatment.

7. The method of claim 1, wherein the method further comprises the steps of:
(a) determining a control (or baseline or reference) ratio of levels of isotope to levels of corresponding non-isotopic atom by measurements of samples taken from the subject before drug administration or taken from a control population of healthy patients or patients who suffer from a Mycobacterium infection; and
(b) comparing the control (or baseline or reference) ratio to ratios of levels of isotope to levels of corresponding non-isotopic atom measured in a sample obtained from a subject after administration of isotopically-labeled drug;
wherein increased ratios of levels of isotope to levels of corresponding non-isotopic atom are indicative of a drug-responsive Mycobacterium infection, and wherein constant or decreasing ratios of levels of isotope to levels of corresponding non-isotopic atom are indicative of the absence of a Mycobacterium infection or of a Mycobacterium infection which is drug resistant.

8. The method of claim 1, wherein levels of isotopically-labeled cleavage products and/or metabolites are measured at intervals of about 1-5 minutes, about 5-10 minutes, about 15-20 minutes, about 25-30 minutes, about 35-40 minutes, about 45-50 minutes, about 55-60 minutes or at regular intervals over the course of about 1, 2, 3, 4 or 5 hours.

9. The method of claim 1, wherein levels of isotopically-labeled cleavage products and/or metabolites are measured using either an infrared spectrometer or an isotope ratio mass spectrometer.

10. The method of claim 1, wherein the subject is suspected of suffering from a Pretomanid or Delaminid-resistant M. tuberculosis infection and wherein the subject is administered ¹⁵N-nitro-PA824 (¹⁵N-nitro-Pretomanid) and ¹⁵N-nitro-OPC67683 (¹⁵N-nitro-Delaminid).

11. The method of claim 1, wherein the subject is suspected of suffering from an ethionamide or prothionamide-resistant M. tuberculosis infection and wherein the subject is administered a composition selected from the group consisting of ¹⁵N-ethionamide; ³³S-ethionamide, ³⁴S-ethionamide, ³⁶S-ethionamide ¹⁵N-prothionamide, ³³S-prothionamide, ³⁴S-prothionamide and ³⁶S-prothionamide.

12. The method of claim 1, wherein the subject is suspected of suffering from an isoniazid-resistant M. tuberculosis infection and wherein the subject is administered ¹⁵N-isoniazid.

13. The method of claim 1, wherein the subject is suspected of suffering from a pyrazinamide-resistant M. tuberculosis infection and wherein the subject is administered a composition selected from the group consisting of ¹⁵N-pyrazinamide ¹³C-pyrazinamide, ¹⁷O-pyrazinamide and ¹⁸O-pyrazinamide.

14. The method of claim 10, wherein the Mycobacterium infection is a Latent tuberculosis infection (LTBI).

15. The method of claim 10, wherein isotopically-labeled drugs are administered by inhalation or orally.

16. (canceled)

17. A composition selected from the group consisting of:
(a) ¹⁵N-nitro-PA824 (¹⁵N-nitro-Pretomanid) and ¹⁵N-nitro-OPC67683 (¹⁵N-nitro-Delaminid);
(b) ¹⁵N-ethionamide; ³³S-ethionamide, ³⁴S-ethionamide and ³⁶S-ethionamide;
(c) ¹⁵N-prothionamide, ³³S-prothionamide, ³⁴S-prothionamide and ³⁶S-prothionamide;
(d) ¹⁵N-pyrazinamide ¹³C-pyrazinamide, ¹⁷O-pyrazinamide and ¹⁸O-pyrazinamide; and
(e) ¹⁵N-isoniazid.

18. (canceled)

19. (canceled)

20. (canceled)

21. (canceled)

22. A diagnostic formulation which can be administered by intratracheal instillation, bronchial instillation, or inhalation, or by an oral, intravenous, intramuscular, intra-arterial, intramedullary, subcutaneous, intraventricular, transdermal, interdermal, rectal, intravaginal, intraperitoneal, topical, transdermal, mucosal, nasal, buccal, enteral, or sublingual route of administration, the formulation comprising:
(a) a composition selected from the group consisting of:
(1) ¹⁵N-nitro-PA824 (¹⁵N-nitro-Pretomanid) and ¹⁵N-nitro-OPC67683 (¹⁵N-nitro-Delaminid);
(2) ¹⁵N-ethionamide; ³³S-ethionamide, ³⁴S-ethionamide and ³⁶S-ethionamide;
(3) ¹⁵N-prothionamide, ³³S-prothionamide, ³⁴S-prothionamide and ³⁶S-prothionamide;
(4) ¹⁵N-pyrazinamide, ¹³C-pyrazinamide, ¹⁷O-pyrazinamide and ¹⁸O-pyrazinamide; and
(5) ¹⁵N-isoniazid; and
(b) one or more pharmaceutically acceptable excipients.

23. The formulation according to claim 22 which is an inhalable dry powder formulation comprising:
(a) a composition selected from the group consisting of:
(1) ¹⁵N-nitro-PA824 (¹⁵N-nitro-Pretomanid) and ¹⁵N-nitro-OPC67683 (¹⁵N-nitro-Delaminid);
(2) ¹⁵N-ethionamide; ³³S-ethionamide, ³⁴S-ethionamide and ³⁶S-ethionamide;
(3) ¹⁵N-prothionamide, ³³S-prothionamide, ³⁴S-prothionamide and ³⁶S-prothionamide;
(4) ¹⁵N-pyrazinamide, ¹³C-pyrazinamide, ¹⁷O-pyrazinamide and ¹⁸O-pyrazinamide; and
(5) ¹⁵N-isoniazid; and
(b) particles of a physiologically acceptable pharmacologically-inert solid carrier.

24. A dry powder inhaler comprising the inhalable dry powder formulation of claim 23.

25. The composition of claim 17 adapted for single dose pulmonary administration to a subject to be diagnosed for the existence or absence of a drug-sensitive M. tuberculosis infection, comprising a diagnostic effective amount of said composition, in combination with a pharmaceutically acceptable excipient, a propellant and optionally, a solvent and a dispersant.

26. The composition of claim 25, wherein the diagnostic effective amount of said composition is present in an amount ranging from about 0.5 mg to about 10 mg each in said composition.

27. The composition of claim 25, wherein said composition is an aerosol.

28. The composition of claim 25, wherein said solvent is a mixture of water and ethanol and said propellant is a CFC substitute fluorinated hydrocarbon.

29. The composition of claim 22, wherein the composition is an enteric composition.

30. The composition of claim 22, wherein the composition contains about 0.5 mg to about 100 mg of isotopically labeled drug.

31. An inhaler containing an aerosol spray formulation which consists of
(a) a diagnostically effective amount of a composition of claim 17; and
(b) a pharmaceutically acceptable dispersant;
wherein the device is metered to disperse an amount of the aerosol formulation by forming a spray that contains a dose of said composition which is effective to diagnose a Mycobacterium infection.

32. A kit comprising one or more compositions of claim 17 in oral or pulmonary dosage form, a collection bag or vial to collect exhaled breaths from a subject, and, optionally, an instruction manual.
